(19) United States
(12) Patent Application Publication
(10) Pub. No.: US 2010/0143364 A1
(43) Pub. Date: Jun. 10, 2010

(54) Chimeric Antibodies, Compositions and Methods for Treating Cocaine-Related Disorders

(75) Inventors: Lee A. Henderson, Ithaca, NY (US); John D. Noti, Athens, PA (US); Wallace R. Fish, Syracuse, NY (US); Brian Miller, Spencer, NY (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: Vybion, Inc., Ithaca, NY (US)

(21) Appl. No.: 12/329,295

(22) Filed: Dec. 5, 2008

(51) Int. Cl. A61K 39/395; C07K 16/00

(52) U.S. Cl. 424/141.1; 530/388.9; 530/389.8

(57) ABSTRACT

The invention relates to antibodies, including chimeric monoclonal antibodies, and fragments thereof, that bind to cocaine. The invention also relates to the use of these or any anti-cocaine antibodies, derivatives or variants in the prevention or treatment of cocaine-related disorders and in the amelioration of one or more symptoms associated with a cocaine-related disorder.

### FIELD OF THE INVENTION

This invention relates generally to chimeric monoclonal antibodies that bind to cocaine, as well as to methods for use thereof.

### BACKGROUND OF THE INVENTION

Cocaine is a powerfully addictive stimulant that directly affects the brain. The pure chemical, cocaine hydrochloride, has been an abused substance for more than 100 years, and coca leaves, the source of cocaine, have been ingested for thousands of years.

Today, cocaine use ranges from occasional use to repeated or compulsive use, with a variety of patterns between these extremes. There is no safe way to use cocaine and any route of administration can lead to absorption of toxic amounts of cocaine, leading to acute cardiovascular or cerebrovascular emergencies that could result in sudden death. Repeated cocaine use by any route of administration can produce dependence, addiction and other adverse health consequences.

Despite decades of basic and clinical research there are currently no medications available to treat cocaine dependence, addiction, overdose or to help prevent relapse. Thus, there exists a need for therapies that target cocaine and treat cocaine-related disorders.

### SUMMARY OF THE INVENTION

The present invention provides monoclonal antibodies, such as chimeric monoclonal antibodies, that specifically bind cocaine. The invention also includes derivatives of the antibodies described herein, such as, for example Fab fragments, that specifically bind cocaine. Exemplary anti-cocaine antibodies include the antibodies referred to herein as chimeric 2E2 antibodies, and variants and/or derivatives thereof. Alternatively, the anti-cocaine antibody or derivative thereof is an antibody or derivative thereof that competes, cross-blocks or otherwise interferes with the binding of the chimeric 2E2 antibodies and derivatives thereof to cocaine. The antibodies are collectively referred to herein as anti-cocaine antibodies. Anti-cocaine antibodies include chimeric antibodies, fully human monoclonal antibodies, as well as humanized monoclonal antibodies.

Anti-cocaine antibodies of the invention also include antibodies that include a heavy chain amino acid sequence that is at least 90%, 91%, 92%, 93%, 94%, 95%, 96%, 97%, 98%, 99% or more identical to the amino acid sequence of SEQ ID NO: 2 and/or a light chain amino acid sequence that is at least 90%, 91%, 92%, 93%, 94%, 95%, 96%, 97%, 98%, 99% or more identical to the amino acid sequence of 6 or 14. Anti-cocaine antibodies of the invention also include antibodies that include a heavy chain amino acid sequence that is encoded by a nucleic acid sequence that is at least 90%, 91%, 92%, 93%, 94%, 95%, 96%, 97%, 98%, 99% or more identical to the nucleotide sequence of SEQ ID NO: 1 or a degenerate nucleic acid sequence that encodes the amino acid sequence of SEQ ID NO: 2, and/or a light chain amino acid sequence that is encoded by a nucleic acid that is at least 90%, 91%, 92%, 93%, 94%, 95%, 96%, 97%, 98%, 99% or more identical to the nucleotide sequence of SEQ ID NO: 5, 13, 17, 18 or 53 or a degenerate nucleic acid sequence that encodes the amino acid sequence SEQ ID NO: 6 or 14.

In some embodiments, the nucleotide sequences encoding the heavy and/or light chain regions of the anti-cocaine antibodies include a leader sequence that is specific for secretion in a particular expression system. For example, the leader sequence is an endogenous leader sequence that is specific for a given cell line. In some embodiments, the leader sequence is an endogenous leader sequence that is specific for expression in a mammalian cell line, such as, e.g., CHO cells. Suitable leader sequences for use with CHO cells are shown within the nucleic acid sequences in SEQ ID NOs: 38 and 45 (nucleotide leader sequences) and within the amino acid sequences in SEQ ID NOs: 39 and 46 (encoded amino acid leader sequences). In some embodiments, the leader sequence is an endogenous leader sequence that is specific for expression in Pischia pastoris. Suitable leader sequences for use with Pischia pastoris are shown within the nucleic acid sequences in SEQ ID NOs: 42 and 43 (nucleotide leader sequences) and within the amino acid sequences in SEQ ID NOs: 41 and 44 (encoded amino acid leader sequences).

In some embodiments, the anti-cocaine antibodies are antibodies in which the nucleotide sequences encoding the heavy and/or light chain regions have been codon-optimized for expression in a particular system. In some embodiments, the nucleotide sequences encoding the heavy and/or light chain regions of the antibody have been codon-optimized for expression in Pischia pastoris. In some embodiments, the nucleotide sequences encoding the heavy and/or light chain regions of the anti-cocaine antibody have been codon-optimized for expression in a mammalian cell line, such as, for example, CHO cells.

The monoclonal antibodies of the present invention use their binding affinity for cocaine and its derivatives to reduce the concentration of cocaine or its derivatives in the brain. The cocaine antagonists, anti-cocaine antibodies and therapeutic formulations of the invention, which include a cocaine antagonist, such as an anti-cocaine antibody of the invention, are used to treat or alleviate one or more cocaine-related disorder(s). As used herein, the term “cocaine-related disorders” includes cocaine dependence, addiction, overdose and/or relapse, and any other disorder resulting in whole or in part from cocaine use. As the site of action of cocaine is in the brain, decreasing the concentrations reaching the brain decreases the probability of dependence, addiction, overdose, and relapse.

Preferably, the three heavy chain complementarity determining regions (CDRs) include an amino acid sequence at least 90%, 91%, 92%, 93%, 94%, 95%, 96%, 97%, 98%, 99% or more identical to each of SDWMNW (SEQ ID NO: 7), NINQDGSEKYYVDSVKG (SEQ ID NO: 8), and ELGP (SEQ ID NO: 9). The light chain CDRs of the ch2E2 antibody include an amino acid sequence at least 90%, 91%, 92%, 93%, 94%, 95%, 96%, 97%, 98%, 99% or more identical to each of RSSTGTITTSNYAN (SEQ ID NO: 10), ATSIRAP (SEQ ID NO: 11), and ALWYNTHYV (SEQ ID NO: 12).

In some embodiments, the VH CDR1 includes at least the amino acid sequence of SDWMNW (SEQ ID NO: 7), the VH CDR2 includes at least the amino acid sequence of NINQDGSEKYYVDSVKG (SEQ ID NO: 8), and the VH CDR3 includes at least the amino acid sequence of ELGP (SEQ ID NO: 9). The VL CDR1 includes at least the amino acid sequence of RSSTGTITTSNYAN (SEQ ID NO: 10), the VL CDR2 includes at least the amino acid sequence of ATSIRAP (SEQ ID NO: 11), and the VL CDR3 includes at least the amino acid sequence of ALWYNTHYV (SEQ ID NO: 12).

In some embodiments, the VH CDR1 includes at least the amino acid sequence of XSDWMNW (SEQ ID NO: 22), where X is selected from serine (S), phenylalanine-serine (F-S), isoleucine-phenylalanine-serine (I-F-S), and phenylalanine-isoleucine-phenylalanine-serine (F-I-F-S); the VH CDR2 includes at least the amino acid sequence of XNINQDGSEKYYVDSVKG (SEQ ID NO: 23), where X is selected from alanine (A), valine-alanine (V-A), tryptophan-valine-alanine (W-V-A) and glutamic acid-tryptophan-valine-alanine (E-W-V-A); and the VH CDR3 includes at least the amino acid sequence of XELGP (SEQ ID NO: 24), where X is selected from lysine (K), alanine-lysine (A-K), cysteine-alanine-lysine (C-A-K) and tyrosine-cysteine-alanine-lysine (Y-C-A-K). The VL CDR1 includes at least the amino acid sequence of XRSSTGTITTSNYAN (SEQ ID NO: 25), where X is selected from cysteine (C), threonine-cysteine, (T-C), leucine-threonine-cysteine (L-T-C), and isoleucine-leucine-threonine-cysteine (I-L-T-C); the VL CDR2 includes at least the amino acid sequence of XATSIRAP (SEQ ID NO: 26), where X is selected from glycine (G), isoleucine-glycine (I-G), leucine-isoleucine-glycine (L-I-G) and glycine-leucine-isoleucine-glycine (G-L-I-G); and the VL CDR3 includes at least the amino acid sequence of XALWYNTHYV (SEQ ID NO: 27), where X is selected from cysteine (C), phenylalanine-cysteine (F-C), tyrosine-phenylalanine-cysteine (Y-F-C) and methionine-tyrosine-phenylalanine-cysteine (M-Y-F-C).

In some embodiments, the VH CDR1 includes at least the amino acid sequence of SDWMNWX (SEQ ID NO: 28), where X is selected from valine (V), valine-arginine (V-R), valine-arginine-glutamine (V-R-Q), and valine-arginine-glutamine-alanine (V-R-Q-A); the VH CDR2 includes at least the amino acid sequence of NINQDGSEKYYVDSVKGX (SEQ ID NO: 29), where X is selected from arginine (R), arginine-phenylalanine (R-F), arginine-phenylalanine-threonine (R-F-T) and arginine-phenylalanine-threonine-isoleucine (R-F-T-I); and the VH CDR3 includes at least the amino acid sequence of ELGPX (SEQ ID NO: 30), where X is selected from tryptophan (W), tryptophan-glycine (W-G), tryptophan-glycine-glutamine (W-G-Q) and tryptophan-glycine-glutamine-glycine (W-G-Q-G). The VL CDR1 includes at least the amino acid sequence of RSSTGTITTSNYANX (SEQ ID NO: 31), where X is selected from tryptophan (W), tryptophan-valine (W-V), tryptophan-valine-glutamine (W-V-Q) and tryptophan-valine-glutamine-lysine (W-V-Q-K); the VL CDR2 includes at least the amino acid sequence of ATSIRAPX (SEQ ID NO: 32), where X is selected from glycine (G), glycine-valine (G-V), glycine-valine-proline (G-V-P) and glycine-valine-proline-valine (G-V-P-V); and the VL CDR3 includes at least the amino acid sequence of ALWYNTHYVX (SEQ ID NO: 33), where X is selected from phenylalanine (F), phenylalanine-glycine (F-G), phenylalanine-glycine-glycine (F-G-G) and phenylalanine-glycine-glycine-glycine (F-G-G-G).

In some embodiments, the VH CDR1 includes at least the amino acid sequence of X₁SDWMNWX₂ (SEQ ID NO: 47), where X₁ is selected from serine (S), phenylalanine-serine (F-S), isoleucine-phenylalanine-serine (I-F-S), and phenylalanine-isoleucine-phenylalanine-serine (F-I-F-S) and where X₂ is selected from valine (V), valine-arginine (V-R), valine-arginine-glutamine (V-R-Q), and valine-arginine-glutamine-alanine (V-R-Q-A); the VH CDR2 includes at least the amino acid sequence of X₁NINQDGSEKYYVDSVKGX₂ (SEQ ID NO: 48), where X₁ is selected from alanine (A), valine-alanine (V-A), tryptophan-valine-alanine (W-V-A) and glutamic acid-tryptophan-valine-alanine (E-W-V-A) and where X₂ is selected from arginine (R), arginine-phenylalanine (R-F), arginine-phenylalanine-threonine (R-F-T) and arginine-phenylalanine-threonine-isoleucine (R-F-T-I); and the VH CDR3 includes at least the amino acid sequence of X₁ELGPX₂ (SEQ ID NO: 49), where X₁ is selected from lysine (K), alanine-lysine (A-K), cysteine-alanine-lysine (C-A-K) and tyrosine-cysteine-alanine-lysine (Y-C-A-K) and where X₂ is selected from tryptophan (W), tryptophan-glycine (W-G), tryptophan-glycine-glutamine (W-G-Q) and tryptophan-glycine-glutamine-glycine (W-G-Q-G).

In some embodiments, the VL CDR1 includes at least the amino acid sequence of X₁RSSTGTITTSNYANX₂ (SEQ ID NO: 50), where X₁ is selected from cysteine (C), threonine-cysteine, (T-C), leucine-threonine-cysteine (L-T-C), and isoleucine-leucine-threonine-cysteine (I-L-T-C) and where X₂ is selected from tryptophan (W), tryptophan-valine (W-V), tryptophan-valine-glutamine (W-V-Q) and tryptophan-valine-glutamine-lysine (W-V-Q-K); the VL CDR2 includes at least the amino acid sequence of X₁ATSIRAPX₂ (SEQ ID NO: 51), where X₁ is selected from glycine (G), isoleucine-glycine (I-G), leucine-isoleucine-glycine (L-I-G) and glycine-leucine-isoleucine-glycine (G-L-I-G) and where X₂ is selected from glycine (G), glycine-valine (G-V), glycine-valine-proline (G-V-P) and glycine-valine-proline-valine (G-V-P-V); and the VL CDR3 includes at least the amino acid sequence of X₁ALWYNTHYVX₂ (SEQ ID NO: 52), where X₁ is selected from cysteine (C), phenylalanine-cysteine (F-C), tyrosine-phenylalanine-cysteine (Y-F-C) and methionine-tyrosine-phenylalanine-cysteine (M-Y-F-C) and where X₂ is selected from phenylalanine (F), phenylalanine-glycine (F-G), phenylalanine-glycine-glycine (F-G-G) and phenylalanine-glycine-glycine-glycine (F-G-G-G).

Preferably, the anti-cocaine antibodies are formatted in an IgG isotype.

The invention also includes derivatives of the antibodies described herein, such as, for example Fab fragments, that specifically bind cocaine. In one embodiment, anti-cocaine antibody fragments of the invention include antibody fragments that include a heavy chain amino acid sequence that is at least 90%, 91%, 92%, 93%, 94%, 95%, 96%, 97%, 98%, 99% or more identical to the amino acid sequence of SEQ ID NO: 2 and/or a light chain amino acid sequence that is at least 90%, 91%, 92%, 93%, 94%, 95%, 96%, 97%, 98%, 99% or more identical to the amino acid sequence of 6 or 14. Anti-cocaine antibody fragments of the invention also include antibody fragments that include a heavy chain amino acid sequence that is encoded by a nucleic acid sequence that is at least 90%, 91%, 92%, 93%, 94%, 95%, 96%, 97%, 98%, 99% or more identical to the nucleotide sequence of SEQ ID NO: 1 or a degenerate nucleic acid sequence that encodes the amino acid sequence of SEQ ID NO: 2, and/or a light chain amino acid sequence that is encoded by a nucleic acid that is at least 90%, 91%, 92%, 93%, 94%, 95%, 96%, 97%, 98%, 99% or more identical to the nucleotide sequence of SEQ ID NO: 5, 13, 17, 18 or 53 or a degenerate nucleic acid sequence that encodes the amino acid sequence SEQ ID NO: 6 or 14.

In some embodiments, the antibody fragments are Fab fragments in which the nucleotide sequences encoding the heavy and/or light chain regions have been codon-optimized for expression in a particular system. In some embodiments, the nucleotide sequences encoding the heavy and/or light chain regions of the Fab fragment have been codon-optimized for expression in Pischia pastoris. In some embodiments, the nucleotide sequences encoding the heavy and/or light chain regions of the Fab fragment have been codon-optimized for expression in a mammalian cell line, such as, for example, CHO cells.

In some embodiments, the nucleotide sequences encoding the heavy and/or light chain regions of the Fab fragments include a leader sequence that is specific for secretion in a particular expression system. For example, the leader sequence is an endogenous leader sequence that is specific for a given cell line. In some embodiments, the leader sequence is an endogenous leader sequence that is specific for expression in a mammalian cell line, such as, e.g., CHO cells. Suitable leader sequences for use with CHO cells are shown within the nucleic acid sequences in SEQ ID NOs: 38 and 45 (nucleotide leader sequences) and within the amino acid sequences in SEQ ID NOs: 39 and 46 (encoded amino acid leader sequences). In some embodiments, the leader sequence is an endogenous leader sequence that is specific for expression in Pischia pastoris. Suitable leader sequences for use with Pischia pastoris are shown within the nucleic acid sequences in SEQ ID NOs: 42 and 43 (nucleotide leader sequences) and within the amino acid sequences in SEQ ID NOs: 41 and 44 (encoded amino acid leader sequences).

The invention also provides antibody fragments, e.g., Fab fragments, that include multiple CDR3 regions. In some embodiments, the antibody fragments, e.g., Fab fragments, include at least two or more copies of a CDR3 region, and specifically bind cocaine. For example, the antibody fragments, e.g., Fab fragments, include 2, 3, 4, 5, 6, 7, 8, 9, 10, 15, 20 or 25 copies of a CDR3 region. In some embodiments, the antibody fragments, e.g., Fab fragments, include multiple copies of a VH CDR3 region and multiple copies of a VL CDR3 region. In some embodiments, the antibody fragments, e.g., Fab fragments, include at least two or more copies of a VH CDR3 region and at least two or more copies of a VL CDR3 region. For example, the antibody fragments, e.g., Fab fragments, include 2, 3, 4, 5, 6, 7, 8, 9, 10, 15, 20 or 25 copies of a VH CDR3 region and 2, 3, 4, 5, 6, 7, 8, 9, 10, 15, 20 or 25 copies of a VL CDR3 region.

In some embodiments, VH CDR3 region includes at least the amino acid sequence of ELGP (SEQ ID NO: 9). In some embodiments, the VH CDR3 region includes at least the amino acid sequence of X₁ELGPX₂ (SEQ ID NO: 49), where X₁ is selected from lysine (K), alanine-lysine (A-K), cysteine-alanine-lysine (C-A-K) and tyrosine-cysteine-alanine-lysine (Y-C-A-K) and where X₂ is selected from tryptophan (W), tryptophan-glycine (W-G), tryptophan-glycine-glutamine (W-G-Q) and tryptophan-glycine-glutamine-glycine (W-G-Q-G). In some embodiments, the VH CDR3 region includes an amino acid sequence that is at least 90%, 91%, 92%, 93%, 94%, 95%, 96%, 97%, 98%, 99% or more identical to ELGP (SEQ ID NO: 9).

In embodiments that include multiple copies of VH CDR3 and VL CDR3 regions, it is understood that any combination of the VH CDR3 and VL CDR3 sequences described herein may be used to produce the fragments.

In some embodiments, the VL CDR3 region includes at least the amino acid sequence of ALWYNTHYV (SEQ ID NO: 12). In some embodiments the VL CDR3 region includes at least the amino acid sequence of X₁ALWYNTHYVX₂ (SEQ ID NO: 52), where X₁ is selected from cysteine (C), phenylalanine-cysteine (F-C), tyrosine-phenylalanine-cysteine (Y-F-C) and methionine-tyrosine-phenylalanine-cysteine (M-Y-F-C) and where X₂ is selected from phenylalanine (F), phenylalanine-glycine (F-G), phenylalanine-glycine-glycine (F-G-G) and phenylalanine-glycine-glycine-glycine (F-G-G-G). In some embodiments, the VL CDR3 region includes an amino acid sequence that is at least 90%, 91%, 92%, 93%, 94%, 95%, 96%, 97%, 98%, 99% or more identical to ALWYNTHYV (SEQ ID NO: 12).

The invention provides isolated monoclonal antibodies or fragments thereof that bind cocaine and includes at least a heavy chain a V_{H} CDR1 region having at least the amino acid sequence of SEQ ID NO: 7, a V_{H} CDR2 region having at least the amino acid sequence of SEQ ID NO: 8, a V_{H} CDR3 region having at least the amino acid sequence of SEQ ID NO: 9, and a light chain that includes at least the light chain constant region of SEQ ID NO: 4, a V_{L} CDR1 region having at least the amino acid sequence of SEQ ID NO: 10, a V_{L} CDR2 region having at least the amino acid sequence of SEQ ID NO: 11, and a V_{L} CDR3 region having at least the amino acid sequence of SEQ ID NO: 12. In some embodiments, the antibodies or fragments thereof includes the heavy chain sequence of SEQ ID NO: 2 and a light chain sequence selected from the group consisting of SEQ ID NOs: 6 and 14. In some embodiments, the antibodies or fragments thereof include a heavy chain sequence encoded by the nucleotide sequence of SEQ ID NO: 1 or a degenerate nucleic acid sequence that encodes the amino acid sequence of SEQ ID NO: 2 and a light chain sequence encoded by a nucleotide sequence selected from the group consisting of SEQ ID NOs: 5, 13, 17, 18 and 53 or a degenerate nucleic acid sequence that encodes an amino acid sequence selected from the group consisting of SEQ ID NOs: 6 and 14. In some embodiments, the antibodies or fragments thereof are IgG isotype. In some embodiments, the antibodies or fragments thereof also include a murine light chain variable region. For example, the murine light chain variable region includes the amino acid sequence of SEQ ID NO: 16.

The invention also provides isolated monoclonal antibodies or fragments thereof that bind cocaine and include the heavy chain sequence of SEQ ID NO: 2 and a light chain sequence selected from the group consisting of SEQ ID NOs: 6 and 14. In some embodiments, the antibodies or fragments thereof include a heavy chain sequence encoded by the nucleotide sequence of SEQ ID NO: 1 or a degenerate nucleic acid sequence that encodes the amino acid sequence of SEQ ID NO: 2 and a light chain sequence encoded by a nucleotide sequence selected from the group consisting of SEQ ID NOs: 5, 13, 17, 18 and 53 or a degenerate nucleic acid sequence that encodes an amino acid sequence selected from the group consisting of SEQ ID NOs: 6 and 14.

The invention provides isolated monoclonal antibodies or fragments thereof that bind cocaine and include heavy chain sequence of SEQ ID NO: 2 and the light chain sequence of SEQ ID NO: 14.

The invention provides pharmaceutical compositions that include any of the anti-cocaine antibodies descried herein and a carrier.

The invention further provides Fab antibody fragments that specifically bind cocaine and include at least two copies of a CDR3 region selected from a VH CDR3 region that includes at least the amino acid sequence of SEQ ID NO: 9, a VL CDR3 region includes at least the amino acid sequence of SEQ ID NO: 12, and combinations thereof. In some embodiments, the Fab fragments also include at least two copies of the VH CDR3 region and at least two copies of the VL CDR3 region.

The invention also provides methods of treating, preventing, alleviating a symptom of, or otherwise mitigating a cocaine-related disorder. In the methods provided herein, the subject to be treated is administered an antagonist of cocaine, e.g., an anti-cocaine monoclonal antibody or a variant and/or derivative thereof. Suitable antagonists of cocaine include any antibody or fragment thereof that inhibits, neutralizes or otherwise interferes with the activity of cocaine, such as, e.g., the anti-cocaine antibodies and derivatives thereof provided herein; small molecule inhibitors; proteins, polypeptides, peptides; protein-, polypeptide- and/or peptide-based antagonists; nucleic acid based antagonists such as siRNA and/or anti-sense RNA, and/or aptamers; and/or fragments thereof that inhibit, neutralize or otherwise interfere with the activity of cocaine.

The antibodies, antibody derivatives, antibody variants and compositions provided herein are useful in treating, preventing or otherwise delaying the progression of a cocaine-related disorder. For example, an anti-cocaine antibody, an anti-cocaine antibody fragment, an anti-cocaine antibody derivative, or other cocaine antagonist of the invention is administered to a subject in need thereof before the onset of a cocaine-related disorder, during the cocaine-related disorder, after an a cocaine-related disorder or any combination thereof.

The subject is suffering from or is predisposed to developing a cocaine-related disorder, such as, for example, cocaine dependence, addiction, overdose and/or relapse, and any other disorder resulting in whole or in part from cocaine use. Preferably, the subject is a mammal, and more preferably, the subject is a human.

The invention provides methods of alleviating a symptom of a cocaine-related disorder by administering an antibody or fragment thereof that binds cocaine to a subject in need thereof in an amount sufficient to alleviate the symptom of the cocaine-related disorder in the subject. In some embodiments, the antibodies or fragments thereof include at least a heavy chain a V_{H} CDR1 region having at least the amino acid sequence of SEQ ID NO: 7, a V_{H} CDR2 region having at least the amino acid sequence of SEQ ID NO: 8, a V_{H} CDR3 region having at least the amino acid sequence of SEQ ID NO: 9, and a light chain that includes at least the light chain constant region of SEQ ID NO: 4, a V_{L} CDR1 region having at least the amino acid sequence of SEQ ID NO: 10, a V_{L} CDR2 region having at least the amino acid sequence of SEQ ID NO: 11, and a V_{L} CDR3 region having at least the amino acid sequence of SEQ ID NO: 12. In some embodiments, the subject is a human. In some embodiments, the antibody or fragment thereof includes a heavy chain sequence encoded by the nucleotide sequence of SEQ ID NO: 1 or a degenerate nucleic acid sequence that encodes the amino acid sequence of SEQ ID NO: 2 and a light chain sequence encoded by a nucleotide sequence selected from the group consisting of SEQ ID NOs: 5, 13, 17, 18 and 53 or a degenerate nucleic acid sequence that encodes an amino acid sequence selected from the group consisting of SEQ ID NOs: 6 and 14. In some embodiments, the antibody or fragment thereof is an IgG isotype. In some embodiments, the antibody or fragment thereof also includes a murine light chain variable region. For example, the murine light chain variable region includes the amino acid sequence of SEQ ID NO: 16.

### DETAILED DESCRIPTION

The present invention provides monoclonal antibodies specific for cocaine, collectively referred to herein as anti-cocaine antibodies. The anti-cocaine antibodies specifically bind cocaine. The invention also includes derivatives of the antibodies described herein, such as, for example Fab fragments, that specifically bind cocaine. As used herein, the terms “specific for”, “specific binding”, “directed against” (and all grammatical variations thereof) are used interchangeably in the context of antibodies that recognize and bind to a cocaine epitope when the equilibrium binding constant (K_{d}) is ≦1 μM, e.g., ≦100 nM, preferably ≦10 nM, and more preferably ≦1 nM. For example, the anti-cocaine antibodies provided herein exhibit a K_{d} in the range approximately between ≦10 nM to about 100 pM.

The anti-cocaine antibodies and variants and/or derivatives thereof are, for example, cocaine antagonists or inhibitors that modulate at least one biological activity of cocaine. Antibodies and antibody derivatives and/or antibody variants with high affinity and specificity for cocaine act as pharmacokinetic antagonists by sequestering cocaine in the peripheral circulation and preventing its entry to the brain. Active immunization of animals with hapten-carrier conjugates has been shown to elicit the production of polyclonal anti-cocaine antibodies with sufficient levels and affinity for cocaine that they can reduce the amount of cocaine entering the brain. Active immunization has also been shown to attenuate the behavioral effects and the priming effect of systemically administered cocaine in rats. Furthermore, the ability of active immunization to produce levels of polyclonal anti-cocaine antibodies in humans that were associated with a decrease in use of cocaine demonstrates the potential efficacy of immunotherapy for cocaine abuse. However, individuals with compromised immune systems (like those who have clinically induced immunosuppression or those who suffer from an infection) often cannot be actively immunized due to the risks of developing a complication from the active immunization. Often, those individuals who are suffering from cocaine-related disorders also have compromised immune systems.

An alternative to active immunization is passive immunization. In passive immunization, a pre-made antibody is given to the individual. While this process is usually short lasting (a few days or even a few weeks), it is much safer and effective for those with compromised immune systems. In addition, using a monoclonal antibody (mAb) with a defined affinity, specificity and dose may be even more efficacious than active immunization. Indeed, passive immunization with non-human anti-cocaine mAbs attenuates the behavioral effects of cocaine and therefore represents an alternative or adjunct to active immunization.

Previously, a murine anti-cocaine antibody (GNC92H2) was generated and demonstrated to have in vivo efficacy in rat models of cocaine addiction. Also, two catalytic murine anti-cocaine mAbs that are designed to reduce blood cocaine levels through its hydrolysis have been generated and characterized. However, non-human sequence anti-cocaine mAbs would be expected to elicit an immune response in humans similar to that elicited by the murine mAb OKT-3 used for immunosuppression for organ transplant procedures. This immune response targets and tries to destroy the non-human mAb thus decreasing or neutralizing the long-term efficacy of such an immunotherapeutic agent. Furthermore, the antibody affinity for cocaine is also a major determinant of clinical efficacy. The affinities of the catalytic mAbs for cocaine are reported to be approximately 220 μM and 55-5,240 μM, while the affinity of the anti-cocaine mAb GNC92H2 is reported to be 200 nM. Therefore, a more efficacious and predominantly human antibody is likely to decrease the probability of inducing a neutralizing immune system response. This theory led to the generation and characterization of monoclonal antibodies which are at least partially generated in transgenic mice that produce human sequence mAbs.

Previous anti-cocaine antibodies were also described in U.S. Patent Application Publication No. 2007/0280934, which describes anti-cocaine antibodies having a murine light chain and a human heavy chain region. These antibodies are referred herein to as “native 2E2 monoclonal antibodies”.

The light chain of these native 2E2 antibodies includes the following murine-derived amino acid sequence:

(SEQ ID NO: 21)

Gln Ala Val Val Ile Gln Glu Ser Ala Leu Thr Thr
Ser Pro Gly Gly Thr Val Ile Leu Thr Cys Arg Ser
Ser Thr Gly Thr Ile Thr Thr Ser Asn Tyr Ala Asn
Trp Val Gln Lys Lys Pro Asn His Val Phe Thr Gly
Leu Ile Gly Ala Thr Ser Ile Arg Ala Pro Gly Val
Pro Val Arg Phe Ser Gly Phe Leu Ile Gly Gly Lys
Ala Ala Leu Thr Ile Thr Gly Ala Gln Thr Glu Asp
Asp Ala Met Tyr Phe Cys Ala Leu Trp Tyr Asn Thr
His Tyr Val Phe Gly Gly Gly Thr Lys Val Thr Val
Leu Gly Gln Pro Lys Ser Thr Pro Thr Leu Thr Val
Phe Pro Pro Ser Ser Glu Glu Leu Lys Glu Asn Lys
Ala Thr Leu Val Cys Leu Ile Ser Asn Phe Ser Pro
Ser Gly Val Thr Val Ala Trp Lys Ala Asn Gly Thr
Pro Ile Thr Gln Gly Val Asp Thr Ser Asn Pro Thr
Lys Glu Gly Asn Lys Phe Met Ala Ser Ser Phe Leu
His Leu Thr Ser Asp Gln Trp Arg Ser His Asn Ser
Phe Thr Cys Gln Val Thr His Glu Gly Asp Thr Val
Glu Lys Ser Leu Ser Pro Ala Glu Cys Leu

The heavy chain of these native 2E2 antibodies includes the following human derived amino acid sequence:

(SEQ ID NO: 2)

Glu Val Gln Leu Val Glu Ser Gly Gly Gly Leu Val
Gln Pro Gly Gly Ser Leu Arg Leu Ser Cys Ala Ala
Ser Gly Phe Ile Phe Ser Ser Asp Trp Met Asn Trp
Val Arg Gln Ala Pro Gly Lys Gly Leu Glu Trp Val
Ala Asn Ile Asn Gln Asp Gly Ser Glu Lys Tyr Tyr
Val Asp Ser Val Lys Gly Arg Phe Thr Ile Ser Arg
Asp Asn Ala Gln Asn Ser Leu Tyr Leu Gln Met Asn
Ser Leu Arg Ala Glu Asp Thr Ala Val Tyr Tyr Cys
Ala Lys Glu Leu Gly Pro Trp Gly Gln Gly Thr Leu
Val Thr Val Ser Ser Ala Ser Thr Lys Gly Pro Ser
Val Phe Pro Leu Ala Pro Ser Ser Lys Ser Thr Ser
Gly Gly Thr Ala Ala Leu Gly Cys Leu Val Lys Asp
Tyr Phe Pro Gla Pro Val Thr Val Ser Trp Asn Ser
Gly Ala Leu Thr Ser Gly Val His Thr Phe Pro Ala
Val Leu Gln Ser Ser Gly Leu Tyr Ser Leu Ser Ser
Val Val Thr Val Pro Ser Ser Ser Leu Gly Thr Gln
Thr Tyr Ile Cys Asn Val Asn His Lys Pro Ser Asn
Thr Lys Val Asp Lys Arg Val Glu Pro Lys Ser Cys
Asp Lys Thr His Thr Cys Pro Pro Cys Pro Ala Pro
Glu Leu Leu Gly Gly Pro Ser Val Phe Leu Phe Pro
Pro Lys Pro Lys Asp Thr Leu Met Ile Ser Arg Thr
Pro Glu Val Thr Cys Val Val Val Asp Val Ser His
Glu Asp Pro Glu Val Lys Phe Asn Trp Tyr Val Asp
Gly Val Glu Val His Asn Ala Lys Thr Lys Pro Arg
Glu Glu Gln Tyr Asn Ser Thr Tyr Arg Val Val Ser
Val Leu Thr Val Leu His Gln Asp Trp Leu Asn Gly
Lys Glu Tyr Lys Cys Lys Val Ser Asn Lys Ala Leu
Pro Ala Pro Ile Glu Lys Thr Ile Ser Lys Ala Lys
Gly Gln Pro Arg Glu Pro Gln Val Tyr Thr Leu Pro
Pro Ser Arg Glu Glu Met Thr Lys Asn Gln Val Ser
Leu Thr Cys Leu Val Lys Gly Phe Tyr Pro Ser Asp
Ile Ala Val Glu Trp Glu Ser Asn Gly Gln Pro Glu
Asn Asn Tyr Lys Thr Thr Pro Pro Val Leu Asp Ser
Asp Gly Ser Phe Phe Leu Tyr Ser Lys Leu Thr Val
Asp Lys Ser Arg Trp Gln Gln Gly Asn Val Phe Ser
Cys Ser Val Met His Glu Ala Leu His Asn His Tyr
Thr Gln Lys Ser Leu Ser Leu Ser Pro Gly

In contrast, the anti-cocaine antibodies, variants and/or derivatives of the invention have a light chain that includes a murine or murine derived variable region and a human or human derived constant region, and a human or human derived heavy chain. The anti-cocaine antibodies, variants and/or derivatives of the invention have at least a similar affinity as the anti-cocaine mAbs that have been previously generated. In addition, these antibodies, variants and/or derivatives have high specificity for cocaine over the major metabolites of cocaine. Therefore, these antibodies, variants and/or derivatives have important physicochemical properties that confer efficacy as a passive immunotherapeutic agent.

The anti-cocaine antibodies, variants and/or derivatives of the invention bind cocaine or a derivative thereof. The anti cocaine antibodies, variants and/or derivatives of the invention have a higher binding specificity for cocaine than for any of the major metabolites or other derivatives of cocaine, such as benzoylecgonine, ecgonine methyl ester, and ecogonine. Other examples of cocaine derivatives include: (−) cocaine; cocaine propyl ester; RTI-128; RTI-66; RTI-160; RTI-192; m-hydroxycocaine; WIN 35,065-2; WIN 35,428; RTI-31; RTI-32; RTI-55; RTI-111; m-hydroxybenzoylecgonine; p-hydroxybenzoylecgonine; RTI-113; tropine; benztropine; 4′,4″-difluoro-3.alpha.-diphenylmethoxytropane; hyoscyamine-N-oxide; methylanisotropine; tropisetronmethiodide; anisodine; scopotamine; scopotamine-N-oxide; methylscopolamine; N-butylscopolamine; (−) pseudococaine; (+) cocaine; norcaine; benzoylnorecgonine; (+) pseudococaine; ecgonidine; exo-6-hydroxytropinone; and methylcocaethylene.

The monoclonal antibodies, variants and/or derivatives of the present invention use their binding affinity for cocaine and its derivatives to reduce the concentration of cocaine or its derivatives in the brain. Infused antibodies, variants and/or derivatives also produce a dramatic dose-dependent increase in plasma cocaine concentrations and a concomitant decrease in the brain cocaine concentrations produced by an i.v. injection of cocaine HCI (0.56 mg/kg). Pharmacokinetic studies show that the normal disappearance of cocaine from plasma is described by a 2-compartment pharmacokinetic model with distribution t_{1/2α} and terminal elimination t_{1/2β} values of 1.9 and 26.1 min, respectively. The anti-cocaine antibodies, variants and/or derivatives of the invention increase the area under the plasma cocaine concentration-time curve (AUC) relative to the AUC in the absence of the anti-cocaine antibodies of the invention. The antibodies, variants and/or derivatives of the present invention decrease cocaine's volume of distribution (l/kg). However, cocaine is still rapidly cleared from plasma and its elimination is described by a single compartment model. The antibodies, variants and/or derivatives also produce a decrease in the cocaine AUC in the brain. Therefore, the effect of the antibodies on plasma and brain cocaine concentrations is predominantly due to a change in the distribution of cocaine with negligible effects on its rate of clearance.

Anti-cocaine antibodies, variants and/or derivatives of the invention include, for example, the heavy chain complementarity determining regions (CDRs) include a VH CDR1 that includes at least the amino acid sequence SDWMNW (SEQ ID NO: 7), a VH CDR2 that includes at least the amino acid sequence NINQDGSEKYYVDSVKG (SEQ ID NO: 8), and a VH CDR3 that includes at least the amino acid sequence ELGP (SEQ ID NO: 9), and the light chain CDRs include a VL CDR1 that includes at least the amino acid sequence RSSTGTITTSNYAN (SEQ ID NO: 10), a VL CDR2 that includes at least the amino acid sequence ATSIRAP (SEQ ID NO: 11), and a VL CDR3 that includes at least the amino acid sequence ALWYNTHYV (SEQ ID NO: 12).

An exemplary anti-cocaine monoclonal antibody is the chimeric light chain 2E2 antibody described herein, also referred to as ch2E2, which includes a human heavy chain and a light chain that includes a murine variable region and a human constant region. As shown below, the ch2E2 antibody includes a human heavy chain (SEQ ID NO. 2) encoded by the nucleic acid sequence of SEQ ID NO: 1 or any other degenerate nucleic acid sequence (i.e., a nucleic acid sequence having codons that are synonymous to the codons presented in SEQ ID NO: 1 according to the universal genetic code) that encodes the amino acid sequence of SEQ ID NO:2, and a chimeric light chain (SEQ ID NO: 6) encoded by the nucleic sequence of SEQ ID NO: 5 or any degenerate nucleic acid sequence that encodes the amino acid sequence of SEQ ID NO: 6. In particular, the ch2E2 antibody includes a human light chain constant region (SEQ ID NO: 4) encoded by the nucleic acid sequence shown in SEQ ID NO: 3 or any degenerate nucleic acid sequence that encodes the amino acid sequence of SEQ ID NO: 4. The CDR sequences include at least the amino acid residues shown in boxes.

ch2E2 antibody: human heavy chain nucleic acid
sequence (SEQ ID NO: 1):
GAGGTGCAGCTGGTGGAGTCTGGGGGAGGCTTGGTCCAGCCTGGGGGGTC
CCTGAGACTCTCCTGTGCAGCCTCTGGATTCATCTTCAGTAGCGATTGGA
TGAACTGGGTCCGCCAGGCTCCAGGGAAAGGGCTGGAGTGGGTGGCCAAC
ATAAACCAAGATGGAAGTGAGAAATACTATGTGGACTCTGTGAAGGGCCG
ATTCACCATCTCCAGAGACAACGCCCAGAACTCACTGTATCTGCAGATGA
ACAGCCTGAGAGCCGAGGACACGGCTGTATATTACTGTGCGAAGGAACTG
GGGCCATGGGGCCAGGGAACCCTGGTCACCGTCTCCTCAGCCTCCACCAA
GGGCCCATCGGTCTTCCCCCTGGCACCCTCCTCCAAGAGCACCTCTGGGG
GCACAGCGGCCCTGGGCTGCCTGGTCAAGGACTACTTCCCCGAACCGGTG
ACGGTGTCGTGGAACTCAGGCGCCCTGACCAGCGGCGTGCACACCTTCCC
GGCTGTCCTACAGTCCTCAGGACTCTACTCCCTCAGCAGCGTGGTGACCG
TGCCCTCCAGCAGCTTGGGCACCCAGACCTACATCTGCAACGTGAATCAC
AAGCCCAGCAACACCAAGGTGGACAAGAGAGTTGAGCCCAAATCTTGTGA
CAAAACTCACACATGCCCACCGTGCCCAGCACCTGAACTCCTGGGGGGAC
CGTCAGTCTTCCTCTTCCCCCCAAAACCCAAGGACACCCTCATGATCTCC
CGGACCCCTGAGGTCACATGCGTGGTGGTGGACGTGAGCCACGAAGACCC
TGAGGTCAAGTTCAACTGGTACGTGGACGGCGTGGAGGTGCATAATGCCA
AGACAAAGCCGCGGGAGGAGCAGTACAACAGCACGTACCGTGTGGTCAGC
GTCCTCACCGTCCTGCACCAGGACTGGCTGAATGGCAAGGAGTACAAGTG
CAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCC
CGGGAGGAGATGACCAAGAACCAGGTCAGCCTGACCTGCCTGGTCAAAGG
CTTCTATCCCAGCGACATCGCCGTGGAGTGGGAGAGCAATGGGCAGCCGG
AGAACAACTACAAGACCACGCCTCCCGTGCTGGACTCCGACGGCTCCTTC
TTCCTCTATAGCAAGCTCACCGTGGACAAGAGCAGGTGGCAGCAGGGGAA
CGTCTTCTCATGCTCCGTGATGCATGAGGCTCTGCACAACCACTACACGC
AGAAGAGCCTCTCCCTGTCTCCGGGTAAA

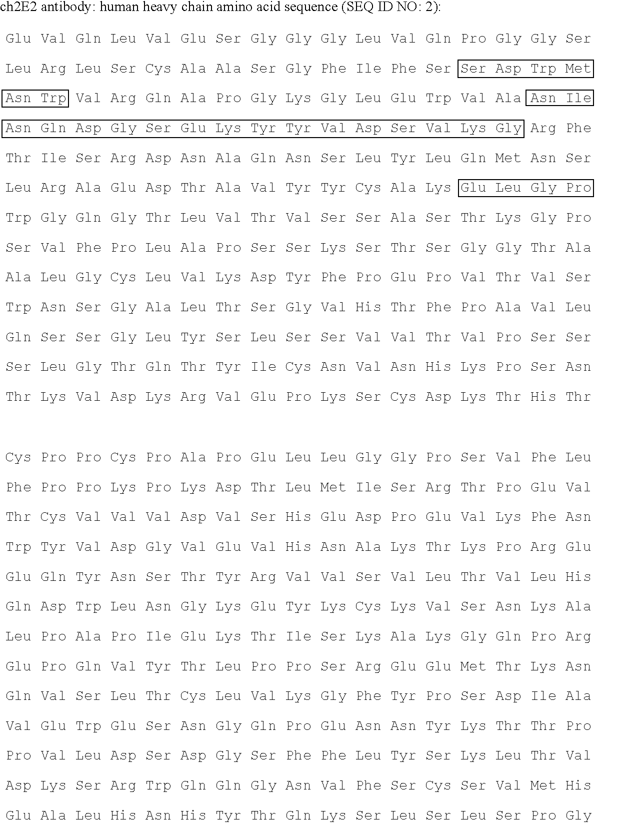

ch2E2 antibody: human light chain constant region
nucleic acid sequence (SEQ ID NO: 3):
GTGGCTGCACCATCTGTCTTCATCTTCCCGCCATCTGATGAGCAGTTGAA
ATCTGGAACTGCCTCTGTTGTGTGCCTGCTGAATAACTTCTATCCCAGAG
AGGCCAAAGTACAGTGGAGGTGGATAACGCCCTCCAATCGGGTAACTCCC
AGGAGAGTGTCACAGAGCAGGACAGCAAGGACAGCACCTACAGCCTCAGC
AGCACCCTGACGCTGAGCAAAGCAGACTACGAGAAACACAAAGTCTACGC
CTGCGAAGTCACCATCAGGGCCTGAGCTCGCCCGTCACAAAGAGCTTCAA
CAGGGGAGAGTGT

ch2E2 antibody: human light chain constant region
amino acid sequence (SEQ ID NO: 4):
VAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSF
NRGEC

ch2E2 antibody: chimeric light chain nucleic acid sequence (SEQ ID NO: 5):

CAGGCTGTTGTGATTCAGGAATCTGCACTCACCACATCACCTGGTGGAAC
AGTCATACTCACTTGTCGCTCAAGTACTGGGACTATTACAACTAGTAACT
ATGCCAACTGGGTCCAAAAAAAACCTAATCATGTATTCACTGGTCTAATA
GGTGCTACCAGCATCCGAGCTCCAGGTGTTCCTGTCAGATTCTCAGGTTT
CCTGATTGGAGGCAAGGCTGCCCTCACCATCACGGGGGCACAGACTGAGG
ATGATGCAATGTATTTCTGTGCTCTATGGTACAACACCCATTATGTTTTC
GGCGGTGGAACCAAGGTCACTGTCCTAGGTCAGCCCAAGGTGGCTGCACC
ATCTGTCTTCATCTTCCCGCCATCTGATGAGCAGTTGAAATCTGGAACTG
CCTCTGTTGTGTGCCTGCTGAATAACTTCTATCCCAGAGAGGCCAAAGTA
CAGTGGAGGTGGATAACGCCCTCCAATCGGGTAACTCCCAGGAGAGTGTC
ACAGAGCAGGACAGCAAGGACAGCACCTACAGCCTCAGCAGCACCCTGAC
GCTGAGCAAAGCAGACTACGAGAAACACAAAGTCTACGCCTGCGAAGTCA
CCATCAGGGCCTGAGCTCGCCCGTCACAAAGAGCTTCAACAGGGGAGAGT
GT

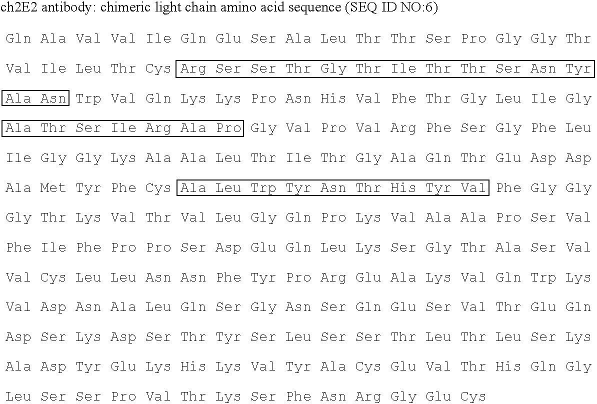

The amino acids encompassing the complementarity determining regions (CDR) are as defined by Chothia et al. and E. A. Kabat et al. (See Chothia, C, et al., Nature 342:877-883 (1989); Kabat, E A, et al., Sequences of Protein of immunological interest, Fifth Edition, US Department of Health and Human Services, US Government Printing Office (1991)). The heavy chain CDRs of the ch2E2 antibody include at least the following sequences: SDWMNW (SEQ ID NO: 7), NINQDGSEKYYVDSVKG (SEQ ID NO: 8), and ELGP (SEQ ID NO: 9). The light chain CDRs of the ch2E2 antibody include at least the following sequences: RSSTGTITTSNYAN (SEQ ID NO: 10), ATSIRAP (SEQ ID NO: 11), and ALWYNTHYV (SEQ ID NO: 12).

Antibody derivatives of the invention, e.g., Fab fragments, include derivatives having the ch2E2 heavy chain, the ch2E2 light chain, and/or a combination of the ch2E2 heavy and light chains. Antibody derivatives, e.g., Fab fragments, include derivatives having at least the CDR regions of the ch2E2 antibody, and a portion of the ch2E2 heavy chain, a portion of the ch2E2 light chain, and/or a combination of a portion of the ch2E2 heavy chain and a portion of the ch2E2 light chain. In some embodiments, the antibody fragments, e.g., Fab fragments, include multiple copies (at least two copies) of the ch2E2 VH CDR3 region, multiple copies (at least two copies) of the ch2E2 VL CDR3 region, and/or a combination of multiple copies (at least two copies) of the ch2E2 VH CDR3 region and multiple copies (at least two copies) of the ch2E2 VL CDR3 region, and at least a portion of the ch2E2 heavy chain, at least a portion of the ch2E2 light chain, and/or a combination of at least a portion of the ch2E2 heavy chain and at least a portion of the ch2E2 light chain. For example, the antibody fragments, e.g., Fab fragments, include 2, 3, 4, 5, 6, 7, 8, 9, 10, 15, 20 or 25 copies of the ch2E2 VH CDR3 region and 2, 3, 4, 5, 6, 7, 8, 9, 10, 15, 20 or 25 copies of the ch2E2 VL CDR3 region. The ch2E2 VH CDR3 region includes at least the amino acid sequence of ELGP (SEQ ID NO: 9). The ch2E2 VL CDR3 region includes at least the amino acid sequence of ALWYNTHYV (SEQ ID NO: 12).

Another exemplary anti-cocaine monoclonal antibody is a variant of the chimeric 2E2 antibody having a modified human light chain constant region, referred to herein as “ch2E2-mutLC” antibodies. In particular, the human light chain constant region of the ch2E2-mutLC antibodies, and variants or derivatives thereof, includes three amino acid mutations (i) an isoleucine (I) to threonine (T) substitution at residue 5 (I5T) of SEQ ID NO: 14, a phenylalanine (F) to serine (S) substitution at residue 67 (F67S) of SEQ ID NO: 14, and a glycine (G) to threonine (T) substitution at residue 102 (G102T) of SEQ ID NO: 14. The ch2E2-mutLC monoclonal antibody includes a human heavy chain and a light chain having a mutated murine light chain variable region and a human light chain constant region. As shown below, the ch2E2-mutLC antibody includes a human heavy chain (SEQ ID NO. 2) encoded by the nucleic acid sequence of SEQ ID NO: 1 or any degenerate nucleic acid sequence that encodes the amino acid sequence of SEQ ID NO:2, and a chimeric light chain (SEQ ID NO: 14) encoded by the nucleic sequence of SEQ ID NO: 13 or SEQ ID NO: 53. The ch2E2-LC antibody includes a human light chain constant region (SEQ ID NO: 4) encoded by the nucleic acid sequence shown in SEQ ID NO: 3 or any degenerate nucleic acid sequence that encodes the amino acid sequence of SEQ ID NO: 4. The ch2E2-mutLC antibody also includes a murine light chain variable region (SEQ ID NO: 16) encoded by the nucleic acid sequence shown in SEQ ID NO: 15 or any degenerate nucleic acid sequence that encodes the amino acid sequence of SEQ ID NO: 16. The CDR sequences include at least the amino acid residues shown in boxes.

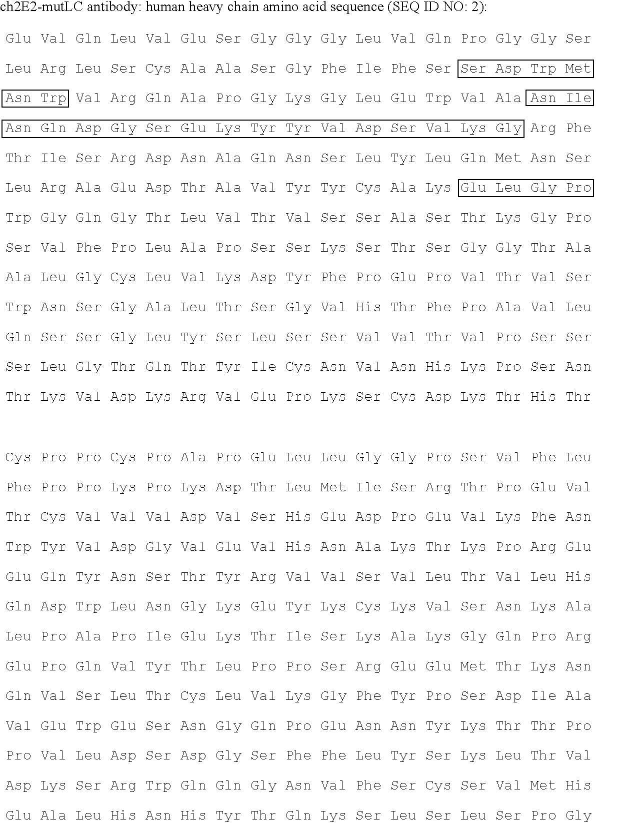

ch2E2-mutLC antibody: light chain nucleic acid
sequence version 1 (SEQ ID NO: 13):
caggctgttgtgacccaggaatctgcactcaccacatcacctggtggaac
agtcatactcacttgtcgctcaagtactgggactattacaactagtaact
atgccaactgggtccaaaaaaaacctaatcatgtattcactggtctaata
ggtgctaccagcatccgagctccaggtgttcctgtcagattctcaggttc
cctgattggaggcaaggctgccctcaccatcacgggggcacagactgagg
atgatgcaatgtatttctgtgctctatggtacaacacccattatgttttc
ggcacaggaaccaaggtcactgtcctaggtcagcccaaggtggctgcacc
atctgtcttcatcttcccgccatctgatgagcagttgaaatctggaactg
cctctgttgtgtgcctgctgaataacttctatcccagagaggccaaagta
cagtggaaggtggataacgccctccaatcgggtaactcccaggagagtgt
cacagagcaggacagcaaggacagcacctacagcctcagcagcaccctga
cgctgagcaaagcagactacgagaaacacaaagtctacgcctgcgaagtc
acccatcagggcctgagctcgcccgtcacaaagagcttcaacaggggaga
gtgt

ch2E2-mutLC antibody: light chain nucleic acid
sequence version 2 (SEQ ID NO: 53):
caggcagtggtgactcaagaatctgcattgacaacctctcccggtggaac
agttatactgacgtgtcgttctagtactggaactataactacctctaact
acgctaactgggttcaaaagaaaccaaatcatgttttcactggcctgatt
ggagcaacatccattagagcccctggagttccagtgagattttctggatc
tttgattggtggtaaggccgctttgactattacgggtgcccaaaccgaag
atgacgcaatgtacttctgcgctctttggtacaacactcattatgttttc
ggtaccggcactaaagttactgtgttgggtcaacctaaagtcgctgcccc
ttcagtctttatctttcctccatccgatgagcagcttaagagtggaactg
cttccgttgtttgtttgcttaacaacttttaccctcgtgaggcaaaagtt
caatggaaagtcgataatgcacttcagtctggaaattctcaagagtctgt
taccgagcaggattctaaggacagtacttattcactgagttcaactttga
ccttgtcaaaggcagattacgaaaagcacaaagtttatgcctgcgaggtc
acgcaccaggggttatcttccccagttacaaagtccttcaatcgagggga
atgttgataagcggccgc

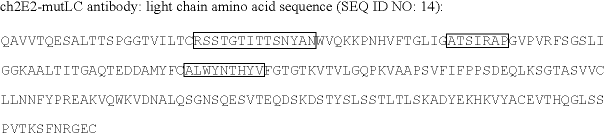

ch2E2-mutLC antibody: murine light chain variable
region nucleic acid sequence (SEQ ID NO: 15):
caggctgttgtgacccaggaatctgcactcaccacatcacctggtggaac
agtcatactcacttgtcgctcaagtactgggactattacaactagtaact
atgccaactgggtccaaaaaaaacctaatcatgtattcactggtctaata
ggtgctaccagcatccgagctccaggtgttcctgtcagattctcaggttc
cctgattggaggcaaggctgccctcaccatcacgggggcacagactgagg
atgatgcaatgtatttctgtgctctatggtacaacacccattatgttttc
ggcacaggaaccaaggtcactgtcctaggtcagcccaag

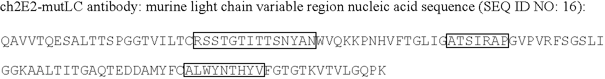

ch2E2-mutLC antibody: human light chain constant
region nucleic acid sequence (SEQ ID NO: 3):
gtggctgcaccatctgtcttcatcttcccgccatctgatgagcagttgaa
atctggaactgcctctgttgtgtgcctgctgaataacttctatcccagag
aggccaaagtacagtggaaggtggataacgccctccaatcgggtaactcc
caggagagtgtcacagagcaggacagcaaggacagcacctacagcctcag
cagcaccctgacgctgagcaaagcagactacgagaaacacaaagtctacg
cctgcgaagtcacccatcagggcctgagctcgcccgtcacaaagagcttc
aacaggggagagtgt

ch2E2-mutLC antibody: human light chain constant
region amino acid sequence (SEQ ID NO: 4)
VAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSF
NRGEC

The amino acids encompassing the complementarity determining regions (CDR) are as defined by Chothia et al. and E. A. Kabat et al. (See Chothia, C, et al., Nature 342:877-883 (1989); Kabat, E A, et al., Sequences of Protein of immunological interest, Fifth Edition, US Department of Health and Human Services, US Government Printing Office (1991)). The heavy chain CDRs of the ch2E2-mutLC antibody include at least the following sequences: SDWMNW (SEQ ID NO: 7), NINQDGSEKYYVDSVKG (SEQ ID NO: 8), and ELGP (SEQ ID NO: 9). The light chain CDRs of the ch2E2-mutLC antibody include at least the following sequences: RSSTGTITTSNYAN (SEQ ID NO: 10), ATSIRAP (SEQ ID NO: 11), and ALWYNTHYV (SEQ ID NO: 12).

Antibody derivatives of the invention, e.g., Fab fragments, include derivatives having the ch2E2-mutLC heavy chain, the ch2E2-mutLC light chain, and/or a combination of the ch2E2-mutLC heavy and light chains. Antibody derivatives, e.g., Fab fragments, include derivatives having at least the CDR regions of the ch2E2-mutLC antibody, and a portion of the ch2E2-mutLC heavy chain, a portion of the ch2E2-mutLC light chain, and/or a combination of a portion of the ch2E2-mutLC heavy chain and a portion of the ch2E2-mutLC light chain. In some embodiments, the antibody fragments, e.g., Fab fragments, include multiple copies (at least two copies) of the ch2E2-mutLC VH CDR3 region, multiple copies (at least two copies) of the ch2E2-mutLC VL CDR3 region, and/or a combination of multiple copies (at least two copies) of the ch2E2-mutLC VH CDR3 region and multiple copies (at least two copies) of the ch2E2-mutLC VL CDR3 region, and at least a portion of the ch2E2-mutLC heavy chain, at least a portion of the ch2E2-mutLC light chain, and/or a combination of at least a portion of the ch2E2-mutLC heavy chain and at least a portion of the ch2E2-mutLC light chain. For example, the antibody fragments, e.g., Fab fragments, include 2, 3, 4, 5, 6, 7, 8, 9, 10, 15, 20 or 25 copies of the ch2E2-mutLC VH CDR3 region and 2, 3, 4, 5, 6, 7, 8, 9, 10, 15, 20 or 25 copies of the ch2E2-mutLC VL CDR3 region. The ch2E2-mutLC VH CDR3 region includes at least the amino acid sequence of ELGP (SEQ ID NO: 9). The ch2E2-mutLC VL CDR3 region includes at least the amino acid sequence of ALWYNTHYV (SEQ ID NO: 12).

Other exemplary anti-cocaine antibodies also include codon-optimized variants of the ch2E2 monoclonal antibody, such as for example, anti-cocaine antibodies having nucleic acid sequences in which particular codons are optimized for expression in a particular host. In some embodiments, these codon-optimized variants also include a modification, e.g., amino acid substitution, in the light chain of the ch2E2 monoclonal antibody.

One exemplary codon-optimized ch2E2 antibody, referred to herein as “ch2E2-mutLC-CO1,” has been optimized for expression in Pichia pastoris and has the following heavy chain and light chain that includes the following nucleic and amino acid sequences or any degenerate nucleic acid sequence that encodes the amino acid sequences shown below. In particular, the human light chain constant region of the ch2E2-mutLC-CO1 antibody includes three amino acid mutations (i) an isoleucine (I) to threonine (T) substitution at residue 5 (I5T) of SEQ ID NO: 14, a phenylalanine (F) to serine (S) substitution at residue 67 (F67S) of SEQ ID NO: 14, and a glycine (G) to threonine (T) substitution at residue 102 (G102T). The CDRs include at least the amino acid residues shown in boxes.

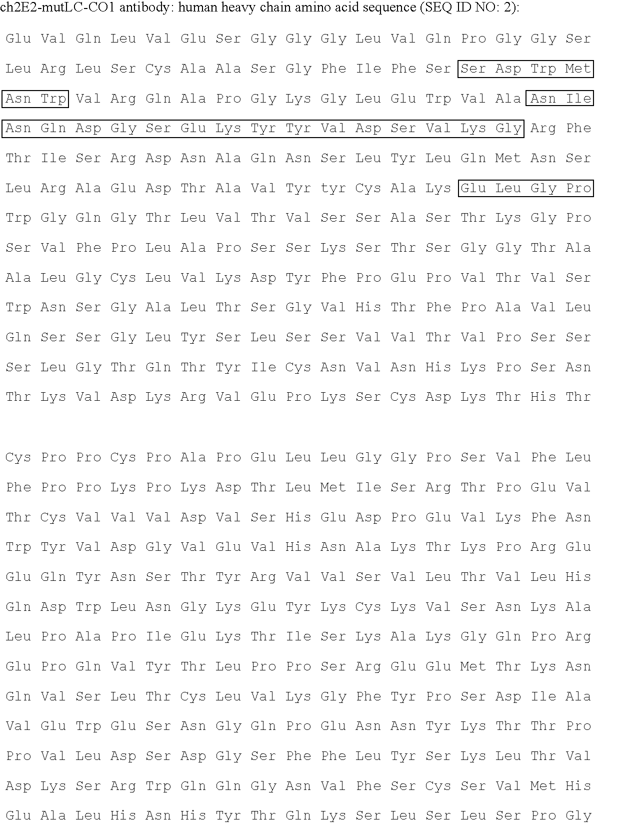

ch2E2-mutLC-CO1 antibody: light chain codon
optimized nucleic acid sequence with murine light
chain variable region and human light chain
constant region (SEQ ID NO: 17):
caggcagtggtgactcaagaatctgcattgacaacctctcccggtggaac
agttatactgacgtgtcgttctagtactggaactataactacctctaact
acgctaactgggttcaaaagaaaccaaatcatgttttcactggcctgatt
ggagcaacatccattagagcccctggagttccagtgagattttctggatc
tttgattggtggtaaggccgctttgactattacgggtgcccaaaccgaag
atgacgcaatgtacttctgcgctctttggtacaacactcattatgttttc
ggtaccggcactaaagttactgtgttgggtcaacctaaagtcgctgcccc
ttcagtctttatctttcctccatccgatgagcagcttaagagtggaactg
cttccgttgtttgtttgcttaacaacttttaccctcgtgaggcaaaagtt
caatggaaagtcgataatgcacttcagtctggaaattctcaagagtctgt
taccgagcaggattctaaggacagtacttattcactgagttcaactttga
ccttgtcaaaggcagattacgaaaagcacaaagtttatgcctgcgaggtc
acgcaccaggggttatcttccccagttacaaagtccttcaatcgagggga
atgt

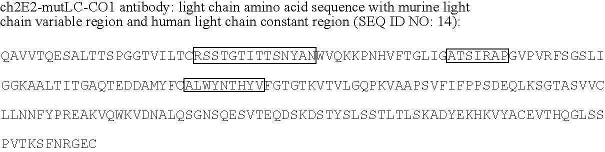

Another exemplary codon-optimized ch2E2 antibody, referred to herein as “ch2E2-mutLC-CO2,” has been optimized for expression in CHO cells and has the following heavy chain sequence and light chain that includes the following nucleic and amino acid sequences or any degenerate nucleic acid sequence that encodes the amino acid sequences shown below. In particular, the human light chain constant region of the ch2E2-mutLC-CO2 antibody includes three amino acid mutations (i) an isoleucine (I) to threonine (T) substitution at residue 5 (I5T) of SEQ ID NO: 14, a phenylalanine (F) to serine (S) substitution at residue 67 (F67S) of SEQ ID NO: 14, and a glycine (G) to threonine (T) substitution at residue 102 (G102T). The CDRs include at least the amino acid residues shown in boxes.

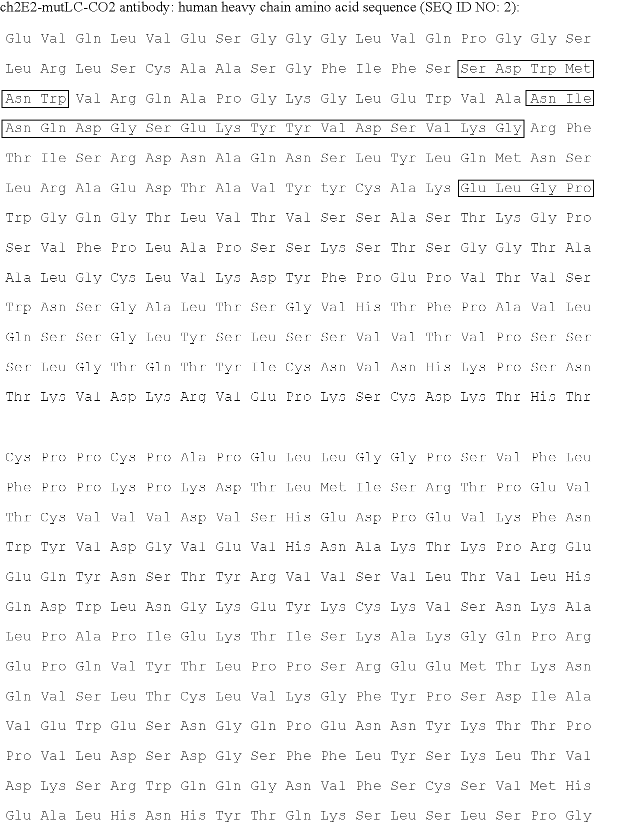

ch2E2-mutLC-CO2 antibody: light chain codon
optimized nucleic acid sequence with murine light
chain variable region and human light chain
constant region (SEQ ID NO: 18):
caggctgttgtgacccaggaatctgcactcaccacatcacctggtggaac
agtcatactcacttgtcgctcaagtactgggactattacaactagtaact
atgccaactgggtccaaaaaaaacctaatcatgtattcactggtctaata
ggtgctaccagcatccgagctccaggtgttcctgtcagattctcaggttc
cctgattggaggcaaggctgccctcaccatcacgggggcacagactgagg
atgatgcaatgtatttctgtgctctatggtacaacacccattatgttttc
ggcacaggaaccaaggtcactgtcctaggtcagcccaaggtggctgcacc
atctgtcttcatcttcccgccatctgatgagcagttgaaatctggaactg
cctctgttgtgtgcctgctgaataacttctatcccagagaggccaaagta
cagtggaaggtggataacgccctccaatcgggtaactcccaggagagtgt
cacagagcaggacagcaaggacagcacctacagcctcagcagcaccctga
cgctgagcaaagcagactacgagaaacacaaagtctacgcctgcgaagtc
acccatcagggcctgagctcgcccgtcacaaagagcttcaacaggggaga
gtgt

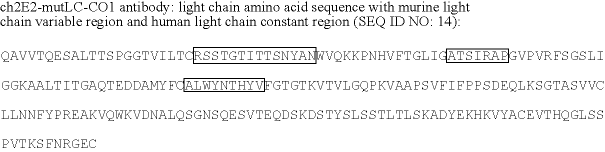

Anti-cocaine antibodies of the invention also include antibodies that include a heavy chain amino acid sequence that is at least 90%, 91%, 92%, 93%, 94%, 95%, 96%, 97%, 98%, 99% or more identical to the amino acid sequence of SEQ ID NO: 2 and/or a light chain amino acid sequence that is at least 90%, 91%, 92%, 93%, 94%, 95%, 96%, 97%, 98%, 99% or more identical to the amino acid sequence of SEQ ID NO: 6 or 14. Anti-cocaine antibodies of the invention also include antibodies that include a heavy chain amino acid sequence that is encoded by a nucleic acid sequence that is at least 90%, 91%, 92%, 93%, 94%, 95%, 96%, 97%, 98%, 99% or more identical to the nucleotide sequence of SEQ ID NO: 1 or a degenerate nucleic acid sequence that encodes the amino acid sequence of SEQ ID NO: 2, and/or a light chain amino acid sequence that is encoded by a nucleic acid that is at least 90%, 91%, 92%, 93%, 94%, 95%, 96%, 97%, 98%, 99% or more identical to the nucleotide sequence of SEQ ID NO: 5, 13, 17, 18 or 53 or a degenerate nucleic acid sequence that encodes the amino acid sequence SEQ ID NO: 6 or 14.

Alternatively, the monoclonal antibody, variant and/or derivative is an antibody that cross-competes, cross-binds or binds to the same portion of cocaine as the anti-cocaine antibodies, variants and/or derivatives of the invention, e.g, ch2E2 and derivatives thereof.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA and oligonucleotide synthesis, as well as tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, delivery and treatment of patients.

As utilized in accordance with the present disclosure, the following terms, unless otherwise indicated, shall be understood to have the following meanings:

As used herein, the term “cocaine-related disorders” includes cocaine dependence, addiction, overdose and/or relapse, and any other disorder resulting in whole or in part from cocaine use. As the site of action of cocaine is in the brain, decreasing the concentrations reaching the brain would be expected to decrease the probability of dependence, addiction, overdose, and relapse.

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and antibodies in an F_{ab} expression library. By “specifically bind” or “immunoreacts with” is meant that the antibody reacts with one or more antigenic determinants of the desired antigen and does not react (i.e., bind) with other polypeptides, chemical targets, small molecules or other targets, or binds at much lower affinity (K_{d}>10⁻⁶) with other polypeptides, chemical targets, small molecules or other targets.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ea., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antigen-binding site,” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

The antibodies provided herein specifically bind a chemical target, i.e., cocaine. An antibody is said to specifically bind an antigen when the dissociation constant is ≦1 μM; e.g., ≦100 nM, preferably ≦10 nM and more preferably ≦1 nM.

As used herein, the terms “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides are quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity, and is equal to the dissociation constant K_{d}. (See, generally, Davies et al. (1990) Annual Rev Biochem 59:439-473). An antibody of the present invention is said to specifically bind to cocaine when the equilibrium binding constant (K_{d}) is ≦1 μM, e.g., ≦100 nM, preferably ≦10 nM, and more preferably ≦1 nM, as measured by assays such as radioligand binding assays or surface plasmon resonance (SPR) or similar assays known to those skilled in the art. For example, the anti-cocaine antibodies provided herein exhibit a K_{d} in the range approximately between ≦10 nM to about 100 pM.

Those skilled in the art will recognize that it is possible to determine, without undue experimentation, if a monoclonal antibody has the same specificity as an anti-cocaine antibody of the invention (e.g., monoclonal antibody ch2E2 and variants thereof) by ascertaining whether the former prevents the latter from binding to cocaine. If the monoclonal antibody being tested competes with an anti-cocaine monoclonal antibody of the invention, as shown by a decrease in binding by the anti-cocaine antibody of the invention, then the two monoclonal antibodies bind to the same, or a closely related, portion of cocaine. Another way to determine whether a monoclonal antibody has the specificity of an anti-cocaine monoclonal antibody of the invention is to pre-incubate the anti-cocaine monoclonal antibody of the invention with cocaine (with which it is normally reactive), and then add the monoclonal antibody being tested to determine if the monoclonal antibody being tested is inhibited in its ability to bind cocaine. If the monoclonal antibody being tested is inhibited then, in all likelihood, it has the same, or functionally equivalent, specificity as the anti-cocaine monoclonal antibody of the invention.

Various procedures known within the art are used for the production of the monoclonal antibodies directed against cocaine, or against derivatives, fragments, analogs homologs or orthologs thereof. (See, e.g., Antibodies: A Laboratory Manual, Harlow E, and Lane D, 1988, Cold Spring Harbor Laboratory Press, Cold Spring Harbor, N.Y., incorporated herein by reference).

Antibodies are purified by well-known techniques, such as affinity chromatography using protein A or protein G. Subsequently, or alternatively, the specific antigen which is the target of the immunoglobulin sought, or the antibody-binding portion thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

In some instances, it may be desirable to modify the antibody of the invention with respect to effector function, so as to enhance, e.g., the effectiveness of the antibody in treating cocaine-related diseases. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)). In a preferred embodiment, the anti-cocaine antibodies of the invention are not modified with respect to effector function.

The invention also includes Fᵥ, F_{ab}, F_{ab′} and F_{(ab′)2} anti-cocaine antibody fragments, single chain anti-cocaine antibodies, bispecific anti-cocaine antibodies and heteroconjugate anti-cocaine antibodies.

Bispecific antibodies are antibodies that have binding specificities for at least two different antigens. In the present case, one of the binding specificities is for cocaine. The second binding target is any other antigen, and in some embodiments, the second binding target is an extracellular target such as a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

Other approaches for generating bispecific antibodies are described, e.g., in WO 96/27011, which is hereby incorporated by reference in its entirety. Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. See e.g., Brennan et al., Science 229:81 (1985), which is hereby incorporated by reference in its entirety.

Additionally, Fab′ fragments can be directly recovered from E. coli and chemically coupled to form bispecific antibodies. See e.g., Shalaby et al., J. Exp. Med. 175:217-225 (1992), which is hereby incorporated by reference in its entirety.

Various techniques for making and isolating bispecific antibody fragments directly from recombinant cell culture have also been described. For example, bispecific antibodies have been produced using leucine zippers. See e.g., Kostelny et al., J. Immunol. 148(5):1547-1553 (1992), which is hereby incorporated by reference in its entirety. The “diabody” technology described by Hollinger et al., Proc. Natl. Acad. Sci. USA 90:6444-6448 (1993), which is hereby incorporated by reference in its entirety, has provided an alternative mechanism for making bispecific antibody fragments. Another strategy for making bispecific antibody fragments by the use of single-chain Fv (sFv) dimers has also been reported. See, Gruber et al., J. Immunol. 152:5368 (1994), which is hereby incorporated by reference in its entirety.

Antibodies with more than two valencies are contemplated. For example, trispecific antibodies can be prepared. See, Tutt et al., J. Immunol. 147:60 (1991).

Heteroconjugate antibodies are also within the scope of the present invention. Heteroconjugate antibodies are composed of two covalently joined antibodies. It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Those of ordinary skill in the art will recognize that a large variety of possible moieties can be coupled to the anti-cocaine antibodies of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling is accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding is achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987). Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl)carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6[3-(2-pyridyldithio)propionamido]hexanoate (Pierce Chem. Co., Cat #21651G); (iv) Sulfo-LC-SPDP (sulfosuccinimidyl 6[3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Preferred polypeptides in accordance with the invention comprise the human heavy chain immunoglobulin molecules presented herein and the human light chain immunoglobulin molecules presented herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

The term oligonucleotide referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the invention are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes Oligonucleotides linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, pp. 101-110 (Volume 5, National Biomedical Research Foundation (1972)) and Supplement 2 to this volume, pp. 1-10. The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotide or amino acid sequences: “reference sequence”, “comparison window”, “sequence identity”, “percentage of sequence identity”, and “substantial identity”. A “reference sequence” is a defined sequence used as a basis for a sequence comparison a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window”, as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2nd Edition, E. S. Golub and D. R. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end the lefthand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity.

Preferably, residue positions which are not identical differ by conservative amino acid substitutions.

Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

As discussed herein, minor variations in the amino acid sequences of antibodies or immunoglobulin molecules are contemplated as being encompassed by the present invention, providing that the variations in the amino acid sequence maintain at least 75% identity, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally-occurring peptide sequence. For example, single or multiple amino acid substitutions (preferably conservative amino acid substitutions) may be made in the naturally-occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion, but where the remaining amino acid sequence is identical to the corresponding positions in the naturally-occurring sequence deduced, for example, from a full length cDNA sequence. Fragments typically are at least 5, 6, 8 or 10 amino acids long, preferably at least 14 amino acids long’ more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to cocaine, under suitable binding conditions or (2) ability to block appropriate cocaine binding. Typically, polypeptide analogs comprise a conservative amino acid substitution (or addition or deletion) with respect to the naturally-occurring sequence. Analogs typically are at least 20 amino acids long, preferably at least 50 amino acids long or longer, and can often be as long as a full-length naturally-occurring polypeptide.

Peptide analogs are commonly used in the pharmaceutical industry as non-peptide drugs with properties analogous to those of the template peptide. These types of non-peptide compound are termed “peptide mimetics” or “peptidomimetics”. Fauchere, J. Adv. Drug Res. 15:29 (1986), Veber and Freidinger TIBS p. 392 (1985); and Evans et al. J. Med. Chem. 30:1229 (1987). Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH-(cis and trans), —COCH₂—, CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992)); for example, by adding internal cysteine residues capable of forming intramolecular disulfide bridges which cyclize the peptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a patient.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present.

Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term patient includes human and veterinary subjects. The term subject includes humans and other mammals.

Design and Generation of Other Therapeutics

In accordance with the present invention and based on the activity of the antibodies that are produced and characterized herein with respect to cocaine, the design of other therapeutic modalities beyond antibody moieties is facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

Knowledge gleaned from the structure of the cocaine molecule and its interactions with other molecules in accordance with the present invention, such as the antibodies of the invention, and others can be utilized to rationally design additional therapeutic modalities. In this regard, rational drug design techniques such as X-ray crystallography, computer-aided (or assisted) molecular modeling (CAMM), quantitative or qualitative structure-activity relationship (QSAR), and similar technologies can be utilized to focus drug discovery efforts. Rational design allows prediction of protein or synthetic structures which can interact with the molecule or specific forms thereof which can be used to modify or modulate the activity of cocaine. Such structures can be synthesized chemically or expressed in biological systems. This approach has been reviewed in Capsey et al. Genetically Engineered Human Therapeutic Drugs (Stockton Press, NY (1988)). Further, combinatorial libraries can be designed and synthesized and used in screening programs, such as high throughput screening efforts.

Therapeutic Administration and Formulations

The monoclonal antibodies, variants and/or derivatives of the present invention use their binding affinity for cocaine and its derivatives to reduce the concentration of cocaine or its derivatives in the brain.

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The cocaine antagonists, anti-cocaine antibodies, variants and/or derivatives and therapeutic formulations of the invention, which include a cocaine antagonist, such as an anti-cocaine antibody of the invention, are used to treat or alleviate one or more cocaine-related disorder(s), including for example, cocaine dependence, addiction, overdose and/or relapse, and any other disorder resulting in whole or in part from cocaine use.

In addition to being a monotherapy, embodiments also include additional co-therapies. In one embodiment, the cocaine antagonist, anti-cocaine antibody, fragment and/or derivative thereof or therapeutic formulation thereof used to cocaine-related disorders is administered in combination with any of a variety of co-therapy agents, or a combination of additional agents. For example, the cocaine antagonist (e.g., anti-cocaine antibody, variant and/or derivative) and additional agent are formulated into a single therapeutic composition, and the cocaine antagonist and additional agent are administered simultaneously. Alternatively, the cocaine antagonist and additional agent are separate from each other, e.g., each is formulated into a separate therapeutic composition, and the cocaine antagonist and the additional agent are administered simultaneously, or the cocaine antagonist and the additional agent are administered at different times during a treatment regimen. For example, the cocaine antagonist (e.g., anti-cocaine antibody) is administered prior to the administration of the additional agent, the cocaine antagonist is administered subsequent to the administration of the additional agent, or the cocaine antagonist and the additional agent are administered in an alternating fashion. As described herein, the cocaine antagonist and additional agent are administered in single doses or in multiple doses.

For example, when treating someone in rehabilitation to prevent relapse, an antibody, variant and/or derivative according to the invention can be administered in conjunction with treatments for withdrawal symptoms (for example, administration of amantadine and propranolol). An antibody, variant and/or derivative according to the invention can be used in conjunction with counseling and other forms of psychotherapy. In addition, it can be used with any antagonist or agonist pharmacotherapies that use compounds for which the antibody does not have substantial affinity. The antibody, variant and/or derivative can also be used with other antibodies that target other drugs of abuse or medicaments.

An antibody, variant and/or derivative according to the present invention may be administered by any suitable route or device. In one embodiment, the antagonist will be administered by injection. The most common form of delivery will be an intravenous injection or infusion.

The anti-cocaine antibodies, variants and/or derivatives and therapeutic formulations thereof are used in methods of treating or alleviating a symptom associated with a cocaine-related disorder. For example, the compositions of the invention are used to treat or alleviate a symptom of any of the anti-cocaine disorders described herein. Symptoms of cocaine abuse include, for example, rapid heartbeat, chest pain, nosebleeds, insomnia, difficulty concentrating, difficulty thinking, inattention, nervousness, paranoid thoughts, and seizures. Symptoms of cocaine overdose include, by way of example, delirium, seizures, respiratory failure, and cardiac arrest. Symptoms of cocaine withdrawal include, e.g., psychological withdrawal symptoms, depression, lack of energy, sore muscles, sweating and shaking.

The cocaine antagonists, such as an anti-cocaine antibodies, variants and/or derivatives, and therapeutic formulations thereof are administered to a subject suffering from a cocaine-related disorder, such as cocaine dependence, addiction, overdose and/or relapse. A subject suffering from a cocaine-related disorder is identified by methods known in the art. For example, subjects are identified using any of a variety of clinical and/or laboratory tests such as, physical examination, radiologic examination and blood, urine and stool analysis to evaluate status.

The antibody, variant and/or derivative is administered in an amount sufficient to treat the cocaine-related disorder. The treatment as used herein encompasses a reduction in clinical symptoms of the disorder and/or elimination of the disorder. Therapeutic amounts will vary based on an individual's age, body weight, symptoms, and the like, and may be determined by one of skill in the art in view of the present disclosure. Initial clinical studies of a cocaine vaccine do provide information about the concentrations of anti-cocaine antibodies required to decrease cocaine use by cocaine abusers. In vaccinated patients the highest mean serum antibody titer would correspond to about 61.4 μg/mL, and a decrease in cocaine use is reported in this cohort as well as cohorts with lower mean antibody titers. As the standard blood volume in a 70 kg person is 2.8 liters, then the quantity of anti-cocaine antibodies that confer efficacy in patients is about 61.4 μg/mL×2,800 ml or about 172 mg/person (about 2.5 mg/kg). This is likely be a minimally effective dose. By comparison, doses of 40 and 120 mg/kg can be safely administered and are efficacious in rodent models. These doses translate to 2,800-8,400 mg in a 70 kg person, almost 20-50-fold higher than may be required to confer efficacy in humans. In addition, studies using drugs like Remicade® (Infliximab), a chimeric monoclonal antibody that has human constant regions and murine variable regions and targets TNF-alpha, also provide information about the dosage of chimeric monoclonal antibodies. Remicade is dosed at a range of about 300-400 mg/70 kg. Furthermore, Fab2 treatments for indications like poisoning, such as DigiFab™ (binds digoxin, used for digitalis overdose and/or poisoning), CroFab® (Crotalidae Polyvalent Antivenin, Fab anti-venom for rattle snake bites or other exposure to snake venom) and Digibind® (binds digoxin, used for digitalis overdose and/or poisoning), also provide useful information about dosing antibodies that bind small molecules. DigiFab™ is dosed at a range of about 400-800 mg, CroFab® is dosed at a range of about 1-5 grams depending on the species of snake, and Digibind® is dosed at a range of about 350-700 mg.

Administration of a cocaine antagonist, such as an anti-cocaine antibody, variant and/or derivative, to a patient is considered successful if any of a variety of laboratory or clinical results is achieved. For example, administration of an anti-cocaine antibody, variant and/or derivative to a patient suffering from cocaine dependence, addiction, overdose and/or relapse is considered successful one or more of the symptoms associated with the disorder is alleviated, reduced, inhibited or does not progress to a further, i.e., worse, state.

Diagnostic Formulations

Antibodies, variants and/or derivatives of the invention are also useful in the detection of cocaine in patient samples and accordingly are useful as diagnostics. For example, the anti-cocaine antibodies, variants and/or derivatives of the invention are used in in vitro assays, e.g., ELISA, to detect cocaine levels in a patient sample.

In one embodiment, an anti-cocaine antibody, variant and/or derivative of the invention is immobilized on a solid support (e.g., the well(s) of a microtiter plate). The immobilized antibody, variant and/or derivative serves as a capture antibody (or fragment) for any cocaine that may be present in a test sample. Prior to contacting the immobilized antibody (or fragment) with a patient sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of cocaine antigen in the test sample is determined by comparison with a standard curve developed from the standard samples.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples, including the experiments conducted and results achieved are provided for illustrative purposes only and are not to be construed as limiting upon the present invention.

### Example 1

### Variants of the ch2E2 Antibodies Having Chimeric Light Chains

Low expression of the native 2E2 anti-cocaine mAb, which includes a human heavy chain and a mouse λ light chain, has been seen from hybridoma cells and in transient transfections. Many factors could cause this phenomena, including a natural incompatibility of a human heavy IgG chain with a mouse light IgG chain. Thus, in the studies described herein, the 1B3 antibody constant region, which is human κ, was spliced to the variable region of the mouse λ light chain. This change increases the expression and/or secretion of the 2E2 antibody while retaining or increasing cocaine affinity.

Briefly, NCBI Protein Blast and other databases were used to determine the probable variable and constant regions of the 2E2 and 1B3 light chains. At the selected junction of the 2E2MuLgt variable/1B3HuLgt constant region, PCR primers were designed to incorporate the first 15 bp of the 1B3HuLgt sequence onto the 3′ end of the 2E2MuLgt chain variable region. Conversely, the final 15 bp of the 2E2MuLgt chain variable region is added onto the 5′-end of the 1B3HuLgt chain constant region. The following PCR primers were used: 2E2MuLgt-linker-3′ (AGATGGTGCAGCCACCTTGGGCTGACCTAG) (SEQ ID NO: 19) and 2E2MuLght-linker-5′ (CTAGGTCAGCCCAAGGTGGCTGCACCATCT) (SEQ ID NO: 20). Using these PCR primers, 2E2MuLgt Variable and 1B3HuLgt Constant intermediate PCR products, with either an EcoRI site or blunt end, containing a 30 bp overlap, were generated and purified. The intermediate PCR products were used in a fusion or “sewing” PCR reaction to produce a full length chimeric 2E2MuLgt Variable/1B3HuLgt Constant products having either EcoRI sites at both ends, or blunt ends.

The resultant chimeric light chain PCR product is then used as a template with primers to the 5′ and 3′ ends of the murine variable and human constant regions, respectively. These 5-′ and 3′-specific primers contain either EcoRI sites (ACGTGAATTCGGCTTACCATGGCCTGG (SEQ ID NO: 34) and ACGTGAATTCTCACTAACACTCTCCCCTG (SEQ ID NO: 35)) or blunt ends (GGCTTACCATGGCCTGGACTTCAC (SEQ ID NO: 36) and TCACTAACACTCTCCCCTGTTGAAGCTC (SEQ ID NO: 37)) to generate the full-length chimeric light chains containing either EcoRI or blunt ends, respectively. The full-length chimeric light chains containing either EcoRI or blunt ends (murine light chain variable region and human light chain constant region) are subsequently cloned into vectors using either an EcoRI site (Vybion pCMV1bd and Stratagene pCMV-Script) or by T/A cloning (Invitrogen pOptiVEC and pcDNA3.3).

The amino acid and nucleic acid sequences for this chimeric light chain ch2E2 antibody, with an endogenous leader sequence specific for expression in CHO cells, are shown below. The amino acid residues in italics below represent the 2E2 mouse λ light chain variable region, and the amino acid residues in bold represent the 1B3 human κ light chain constant region. The DNA sequence was derived from sequencing of cDNA clones.

| | |
|---|---|
| atggcctggacttcacttatactctctctcctggctctctgctcaggagccagt | (SEQ ID NO: 38) |
| M A W T S L I L S L L A L C S G A S | (SEQ ID NO: 39) |
| tcccaggctgttgtgattcaggaatctgcactcaccacatcacctggtggaacagtcata | |
| S Q A V V I Q E S A L T T S P G G T V I | |
| ctcacttgtcgctcaagtactgggactattacaactagtaactatgccaactgggtccaa | |
| L T C R S S T G T I T T S N Y A N W V Q | |
| aaaaaacctaatcatgtattcactggtctaataggtgctaccagcatccgagctccaggt | |
| K K P N H V F T G L I G A T S I R A P G | |
| gttcctgtcagattctcaggtttcctgattggaggcaaggctgccctcaccatcacgggg | |
| V P V R F S G F L I G G K A A L T I T G | |
| gcacagactgaggatgatgcaatgtatttctgtgctctatggtacaacacccattatgtt | |
| A Q T E D D A M Y F C A L W Y N T H Y V | |
| ttcggcggtggaaccaaggtcactgtcctaggtcagcccaaggtggctgcaccatctgtc | |
| F G G G T K V T V L G Q P K V A A P S V | |
| ttcatcttcccgccatctgatgagcagttgaaatctggaactgcctctgttgtgtgcctg | |
| F I F P P S D E Q L K S G T A S V V C L | |
| ctgaataacttctatcccagagaggccaaagtacagtggaaggtggataacgccctccaa | |
| L N N F Y P R E A K V Q W K V D N A L Q | |
| tcgggtaactcccaggagagtgtcacagagcaggacagcaaggacagcacctacagcctc | |
| S G N S Q E S V T E Q D S K D S T Y S L | |
| agcagcaccctgacgctgagcaaagcagactacgagaaacacaaagtctacgcctgcgaa | |
| S S T L T L S K A D Y E K H K V Y A C E | |
| Gtcacccatcagggcctgagctcgcccgtcacaaagagcttcaacaggggagagtgt | |
| V T H Q G L S S P V T K S F N R G E C | |

### Example 2

### Codon-Optimized Variants of the ch2E2 Antibodies

An in silico analysis of the amino acid primary structure and possible T-cell epitopes of the chimeric 2E2 IgG light chain described above in SEQ ID NO: 6 (i.e., 2E2 murine variable region with 1B3 human κ constant region) indicated that two amino acid residues, Kabat I5 and F67 (both hydrophobic), could possibly cause structural problems since the normally seen, and germline, sequences showed hydrophilic amino acids (T and S, respectively) in those positions. Also, a known TCED (T-cell epitope) was detected in the Framework 4 region of the 2E2 MuVar region. This latter amino acid sequence showed divergence from the J germline sequence in possessing a “GGG” triplet that usually presents as “GTG”. Thus, the middle G to T is changed to result in the T-cell epitope becoming identical to a human JL1, i.e., YVGTGTKVTVL (SEQ ID NO: 40).

In the variants described herein, codons were selected so as to avoid creating restriction sites within the optimized sequences. The 2E2 Fab light and heavy sequences optimized by DNA2.0 for Pichia pastoris were assessed for the codons used for T and S. The codons used were identical in weighting to those found on the Kazusa Codon Usage Database. For the P. pastoris pPIC3.5K and pPIC9K constructs provided below, ACT and ACC are used for T (threonine), (the use of ACC creates a Acc65I site which is an alternative linearization restriction enzyme (RE) for pPICZα), and TCT is used for the one change to S (serine) (the use of TCC would create a BamHI site, which is used for cloning). The ch2E2-LC for mammalian expression is changed at the same three (3) codons by using the Kazusa Cricetulus griseus (Chinese hamster) codon usage table. Thus, ACC and ACA are used for T and TCC for S.

Sequences for Pichia are those synthesized by DNA2.0 with codon optimized heavy and chimeric light chains. Unmarked portions of pPIC3.5K and pPIC9K sequences are AOX1 promoter, AOX1 translation termination and α-mating factor sequence regions. All nucleotide and amino acid changes are shown in boxes.

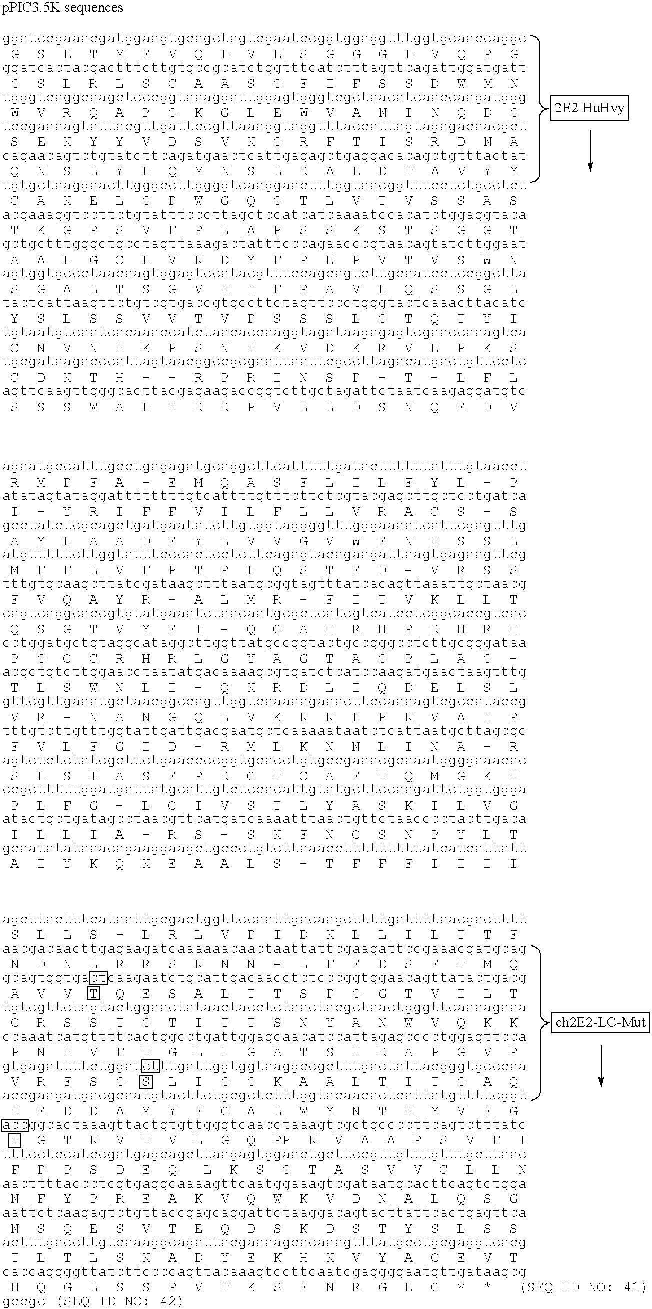

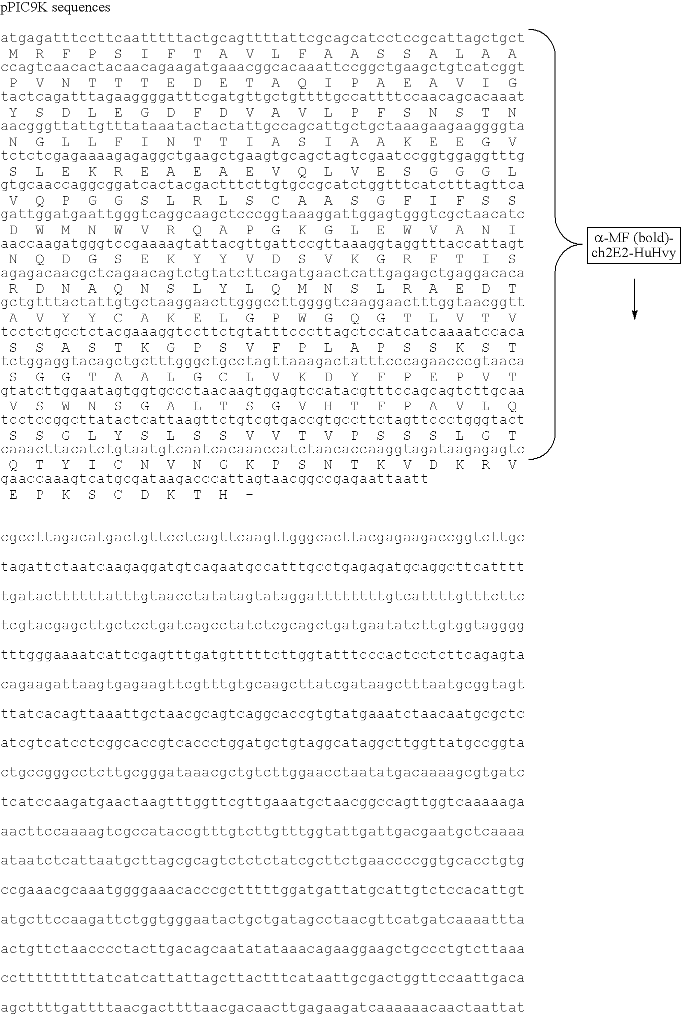

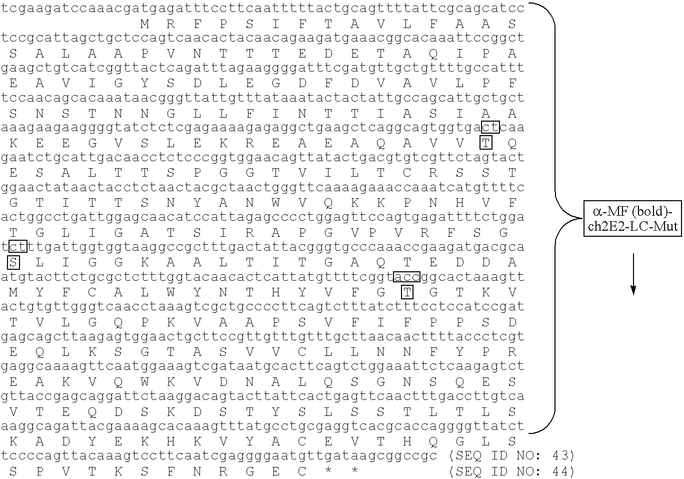

| | |
|---|---|
| CHO Cell sequences | |
| gcggccgcggcttaccatggcctggacttcacttatactctctctcctggctctctgctca | (SEQ ID NO: 45) |
| M A W T S L I L S L L A L C S | (SEQ ID NO: 46) |
| ggagccagttcccaggctgttgtgacccaggaatctgcactcaccacatcacctggtgga | |
| G A S S Q A V V T Q E S A L T T S P G G | |
| acagtcatactcacttgtcgctcaagtactgggactattacaactagtaactatgccaac | |
| T V I L T C R S S T G T I T T S N Y A N | |
| tgggtccaaaaaaaacctaatcatgtattcactggtctaataggtgctaccagcatccga | |
| W V Q K K P N H V F T G L I G A T S I R | |
| gctccaggtgttcctgtcagattctcaggttccctgattggaggcaaggctgccctcacc | |
| A P G V P V R F S G S L I G G K A A L T | |
| atcacgggggcacagactgaggatgatgcaatgtatttctgtgctctatggtacaacacc | |
| I T G A Q T E D D A M Y F C A L W Y N T | |
| cattatgttttcggcacaggaaccaaggtcactgtcctaggtcagcccaaggtggctgca | |
| H Y V F G T G T K V T V L G Q P K V A A | |
| ccatctgtcttcatcttcccgccatctgatgagcagttgaaatctggaactgcctctgtt | |
| P S V F I F P P S D E Q L K S G T A S V | |
| gtgtgcctgctgaataacttctatcccagagaggccaaagtacagtggaaggtggataac | |
| V C L L N N F Y P R E A K V Q W K V D N | |
| gccctccaatcgggtaactcccaggagagtgtcacagagcaggacagcaaggacagcacc | |
| A L Q S G N S Q E S V T E Q D S K D S T | |
| tacagcctcagcagcaccctgacgctgagcaaagcagactacgagaaacacaaagtctac | |
| Y S L S S T L T L S K A D Y E K H K V Y | |
| gcctgcgaagtcacccatcagggcctgagctcgcccgtcacaaagagcttcaacagggga | |
| A C E V T H Q G L S S P V T K S F N R G | |
| Gagtgttagtgagaattc | |
| E C * * | |

All sequences were checked for absence of restriction enzyme sites necessary for either cloning or linearization, and none were present.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated monoclonal antibody or fragment thereof that binds cocaine, wherein said antibody or fragment thereof comprises a heavy chain a V_{H} CDR1 region comprising the amino acid sequence of SEQ ID NO: 7, a V_{H} CDR2 region comprising the amino acid sequence of SEQ ID NO: 8, a V_{H} CDR3 region comprising the amino acid sequence of SEQ ID NO: 9, and a light chain comprising the light chain constant region of SEQ ID NO: 4, a V_{L} CDR1 region comprising the amino acid sequence of SEQ ID NO: 10, a V_{L} CDR2 region comprising the amino acid sequence of SEQ ID NO: 11, and a V_{L} CDR3 region comprising the amino acid sequence of SEQ ID NO: 12.

2. The antibody or fragment of claim 1, wherein said antibody or fragment thereof comprises the heavy chain sequence of SEQ ID NO: 2 and a light chain sequence selected from the group consisting of SEQ ID NOs: 6 and 14.

3. The antibody or fragment of claim 1, wherein said antibody or fragment thereof comprises a heavy chain sequence encoded by the nucleotide sequence of SEQ ID NO: 1 or a degenerate nucleic acid sequence that encodes the amino acid sequence of SEQ ID NO: 2 and a light chain sequence encoded by a nucleotide sequence selected from the group consisting of SEQ ID NOs: 5, 13, 17, 18 and 53 or a degenerate nucleic acid sequence that encodes an amino acid sequence selected from the group consisting of SEQ ID NOs: 6 and 14.

4. The antibody or fragment of claim 1, wherein said antibody or fragment thereof is an IgG isotype.

5. The antibody or fragment of claim 1, wherein said antibody or fragment thereof further comprises a murine light chain variable region.

6. The antibody or fragment of claim 5, wherein said murine light chain variable region comprises the amino acid sequence of SEQ ID NO: 16.

7. An isolated monoclonal antibody or fragment thereof that binds cocaine, wherein said antibody or fragment thereof comprises the heavy chain sequence of SEQ ID NO: 2 and a light chain sequence selected from the group consisting of SEQ ID NOs: 6 and 14.

8. The antibody or fragment of claim 7, wherein said antibody or fragment thereof comprises a heavy chain sequence encoded by the nucleotide sequence of SEQ ID NO: 1 or a degenerate nucleic acid sequence that encodes the amino acid sequence of SEQ ID NO: 2 and a light chain sequence encoded by a nucleotide sequence selected from the group consisting of SEQ ID NOs: 5, 13, 17, 18 and 53 or a degenerate nucleic acid sequence that encodes an amino acid sequence selected from the group consisting of SEQ ID NOs: 6 and 14.

9. An isolated monoclonal antibody or fragment thereof that binds cocaine, wherein said antibody or fragment thereof comprises the heavy chain sequence of SEQ ID NO: 2 and the light chain sequence of SEQ ID NO: 14.

10. A pharmaceutical composition comprising the antibody or fragment of claim 1 and a carrier.

11. A method of alleviating a symptom of a cocaine-related disorder, the method comprising administering an antibody or fragment thereof that binds cocaine to a subject in need thereof in an amount sufficient to alleviate the symptom of the cocaine-related disorder in the subject, wherein the antibody or fragment thereof comprises a heavy chain a V_{H} CDR1 region comprising the amino acid sequence of SEQ ID NO: 7, a V_{H} CDR2 region comprising the amino acid sequence of SEQ ID NO: 8, a V_{H} CDR3 region comprising the amino acid sequence of SEQ ID NO: 9, and a light chain comprising the light chain constant region of SEQ ID NO: 4, a V_{L} CDR1 region comprising the amino acid sequence of SEQ ID NO: 10, a V_{L} CDR2 region comprising the amino acid sequence of SEQ ID NO: 11, and a V_{L} CDR3 region comprising the amino acid sequence of SEQ ID NO: 12.

12. The method of claim 11, wherein said subject is a human.

13. The method of claim 11, wherein said antibody or fragment thereof comprises a heavy chain sequence encoded by the nucleotide sequence of SEQ ID NO: 1 or a degenerate nucleic acid sequence that encodes the amino acid sequence of SEQ ID NO: 2 and a light chain sequence encoded by a nucleotide sequence selected from the group consisting of SEQ ID NOs: 5, 13, 17, 18 and 53 or a degenerate nucleic acid sequence that encodes an amino acid sequence selected from the group consisting of SEQ ID NOs: 6 and 14.

14. The method of claim 11, wherein said antibody or fragment thereof is an IgG isotype.

15. The method of claim 11, wherein said antibody or fragment thereof further comprises a murine light chain variable region.

16. The method of claim 11, wherein said murine light chain variable region comprises the amino acid sequence of SEQ ID NO: 16.

17. An Fab antibody fragment comprising at least two copies of a CDR3 region selected from a VH CDR3 region that includes at least the amino acid sequence of SEQ ID NO: 9, a VL CDR3 region includes at least the amino acid sequence of SEQ ID NO: 12, and combinations thereof, wherein said Fab fragment specifically binds cocaine.

18. The Fab antibody fragment of claim 17, wherein said fragment comprises at least two copies of the VH CDR3 region and at least two copies of the VL CDR3 region.


SEQUENCE LISTING (53 sequences)

SEQ ID NO: 1 - DNA, 1329 bp, Homo sapiens
gaggtgcagctggtggagtctgggggaggcttggtccagcctggggggtccctgagactctcctgtgcagcctctggattcatcttcagtagcgattggatgaactgggtccgccaggctccagggaaagggctggagtgggtggccaacataaaccaagatggaagtgagaaatactatgtggactctgtgaagggccgattcaccatctccagagacaacgcccagaactcactgtatctgcagatgaacagcctgagagccgaggacacggctgtatattactgtgcgaaggaactggggccatggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 2 - protein, 442 aa, Homo sapiens
EVQLVESGGGLVQPGGSLRLSCAASGFIFSSDWMNWVRQAPGKGLEWVANINQDGSEKYYVDSVKGRFTISRDNAQNSLYLQMNSLRAEDTAVYYCAKELGPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - DNA, 313 bp, Homo sapiens
gtggctgcaccatctgtcttcatcttcccgccatctgatgagcagttgaaatctggaactgcctctgttgtgtgcctgctgaataacttctatcccagagaggccaaagtacagtggaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcaccatcagggcctgagctcgcccgtcacaaagagcttcaacaggggagagtgt

SEQ ID NO: 4 - protein, 105 aa, Homo sapiens [sequence omitted]

SEQ ID NO: 5 - DNA, 652 bp, Artificial Sequence
  chemically synthesized
caggctgttgtgattcaggaatctgcactcaccacatcacctggtggaacagtcatactcacttgtcgctcaagtactgggactattacaactagtaactatgccaactgggtccaaaaaaaacctaatcatgtattcactggtctaataggtgctaccagcatccgagctccaggtgttcctgtcagattctcaggtttcctgattggaggcaaggctgccctcaccatcacgggggcacagactgaggatgatgcaatgtatttctgtgctctatggtacaacacccattatgttttcggcggtggaaccaaggtcactgtcctaggtcagcccaaggtggctgcaccatctgtcttcatcttcccgccatctgatgagcagttgaaatctggaactgcctctgttgtgtgcctgctgaataacttctatcccagagaggccaaagtacagtggaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcaccatcagggcctgagctcgcccgtcacaaagagcttcaacaggggagagtgt

SEQ ID NO: 6 - protein, 218 aa, Artificial Sequence
  chemically synthesized
QAVVIQESALTTSPGGTVILTCRSSTGTITTSNYANWVQKKPNHVFTGLIGATSIRAPGVPVRFSGFLIGGKAALTITGAQTEDDAMYFCALWYNTHYVFGGGTKVTVLGQPKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 6 aa, Homo sapiens
SDWMNW

SEQ ID NO: 8 - protein, 17 aa, Homo sapiens
NINQDGSEKYYVDSVKG

SEQ ID NO: 9 - protein, 4 aa, Homo sapiens
ELGP

SEQ ID NO: 10 - protein, 14 aa, Mus musculus
RSSTGTITTSNYAN

SEQ ID NO: 11 - protein, 7 aa, Mus musculus
ATSIRAP

SEQ ID NO: 12 - protein, 9 aa, Mus musculus
ALWYNTHYV

SEQ ID NO: 13 - DNA, 654 bp, Artificial Sequence
  chemically synthesized
caggctgttgtgacccaggaatctgcactcaccacatcacctggtggaacagtcatactcacttgtcgctcaagtactgggactattacaactagtaactatgccaactgggtccaaaaaaaacctaatcatgtattcactggtctaataggtgctaccagcatccgagctccaggtgttcctgtcagattctcaggttccctgattggaggcaaggctgccctcaccatcacgggggcacagactgaggatgatgcaatgtatttctgtgctctatggtacaacacccattatgttttcggcacaggaaccaaggtcactgtcctaggtcagcccaaggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 14 - protein, 218 aa, Artificial Sequence
  chemically synthesized
QAVVTQESALTTSPGGTVILTCRSSTGTITTSNYANWVQKKPNHVFTGLIGATSIRAPGVPVRFSGSLIGGKAALTITGAQTEDDAMYFCALWYNTHYVFGTGTKVTVLGQPKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 339 bp, Artificial Sequence
  chemically synthesized
caggctgttgtgacccaggaatctgcactcaccacatcacctggtggaacagtcatactcacttgtcgctcaagtactgggactattacaactagtaactatgccaactgggtccaaaaaaaacctaatcatgtattcactggtctaataggtgctaccagcatccgagctccaggtgttcctgtcagattctcaggttccctgattggaggcaaggctgccctcaccatcacgggggcacagactgaggatgatgcaatgtatttctgtgctctatggtacaacacccattatgttttcggcacaggaaccaaggtcactgtcctaggtcagcccaag

SEQ ID NO: 16 - protein, 113 aa, Artificial Sequence
  chemically synthesized
QAVVTQESALTTSPGGTVILTCRSSTGTITTSNYANWVQKKPNHVFTGLIGATSIRAPGVPVRFSGSLIGGKAALTITGAQTEDDAMYFCALWYNTHYVFGTGTKVTVLGQPK

SEQ ID NO: 17 - DNA, 654 bp, Artificial Sequence
  chemically synthesized
caggcagtggtgactcaagaatctgcattgacaacctctcccggtggaacagttatactgacgtgtcgttctagtactggaactataactacctctaactacgctaactgggttcaaaagaaaccaaatcatgttttcactggcctgattggagcaacatccattagagcccctggagttccagtgagattttctggatctttgattggtggtaaggccgctttgactattacgggtgcccaaaccgaagatgacgcaatgtacttctgcgctctttggtacaacactcattatgttttcggtaccggcactaaagttactgtgttgggtcaacctaaagtcgctgccccttcagtctttatctttcctccatccgatgagcagcttaagagtggaactgcttccgttgtttgtttgcttaacaacttttaccctcgtgaggcaaaagttcaatggaaagtcgataatgcacttcagtctggaaattctcaagagtctgttaccgagcaggattctaaggacagtacttattcactgagttcaactttgaccttgtcaaaggcagattacgaaaagcacaaagtttatgcctgcgaggtcacgcaccaggggttatcttccccagttacaaagtccttcaatcgaggggaatgt

SEQ ID NO: 18 - DNA, 654 bp, Artificial Sequence; identical to SEQ ID NO: 13
  chemically synthesized

SEQ ID NO: 19 - DNA, 30 bp, Artificial Sequence
  chemically synthesized
agatggtgcagccaccttgggctgacctag

SEQ ID NO: 20 - DNA, 30 bp, Artificial Sequence
  chemically synthesized
ctaggtcagcccaaggtggctgcaccatct

SEQ ID NO: 21 - protein, 214 aa, Mus musculus
QAVVIQESALTTSPGGTVILTCRSSTGTITTSNYANWVQKKPNHVFTGLIGATSIRAPGVPVRFSGFLIGGKAALTITGAQTEDDAMYFCALWYNTHYVFGGGTKVTVLGQPKSTPTLTVFPPSSEELKENKATLVCLISNFSPSGVTVAWKANGTPITQGVDTSNPTKEGNKFMASSFLHLTSDQWRSHNSFTCQVTHEGDTVEKSLSPAECL

SEQ ID NO: 22 - protein, 7 aa, Homo sapiens
  MISC_FEATURE (1)..(1): where X is selected from serine, phenylalanine-serine, isoleucine-phenylalanine-serine, and phenylalanine-isoleucine-phenylalanine-serine
XSDWMNW

SEQ ID NO: 23 - protein, 18 aa, Homo sapiens
  MISC_FEATURE (1)..(1): where X is selected from alanine, valine-alanine, tryptophan-valine-alanine and glutamic acid-tryptophan-valine-alanine
XNINQDGSEKYYVDSVKG

SEQ ID NO: 24 - protein, 5 aa, Homo sapiens
  MISC_FEATURE (1)..(1): where X is selected from lysine, alanine-lysine, cysteine-alanine-lysine and tyrosine-cysteine-alanine-lysine
XELGP

SEQ ID NO: 25 - protein, 15 aa, Mus musculus
  MISC_FEATURE (1)..(1): where X is selected from cysteine, threonine-cysteine, leucine-threonine-cysteine, and isoleucine-leucine-threonine-cysteine
XRSSTGTITTSNYAN

SEQ ID NO: 26 - protein, 8 aa, Mus musculus
  MISC_FEATURE (1)..(1): where X is selected from glycine, isoleucine-glycine, leucine-isoleucine-glycine and glycine-leucine-isoleucine-glycine
XATSIRAP

SEQ ID NO: 27 - protein, 10 aa, Mus musculus
  MISC_FEATURE (1)..(1): where X is selected from cysteine, phenylalanine-cysteine, tyrosine-phenylalanine-cysteine and methionine-tyrosine-phenylalanine-cysteine
XALWYNTHYV

SEQ ID NO: 28 - protein, 7 aa, Homo sapiens
  MISC_FEATURE (7)..(7): where X is selected from valine, valine-arginine, valine-arginine-glutamine, and valine-arginine-glutamine-alanine
SDWMNWX

SEQ ID NO: 29 - protein, 18 aa, Homo sapiens
  MISC_FEATURE (18)..(18): where X is selected from arginine, arginine-phenylalanine, arginine-phenylalanine-threonine and arginine-phenylalanine-threonine-isoleucine
NINQDGSEKYYVDSVKGX

SEQ ID NO: 30 - protein, 5 aa, Homo sapiens
  MISC_FEATURE (5)..(5): where X is selected from tryptophan, tryptophan-glycine, tryptophan-glycine-glutamine and tryptophan-glycine-glutamine-glycine
ELGPX

SEQ ID NO: 31 - protein, 15 aa, Mus musculus
  MISC_FEATURE (15)..(15): where X is selected from tryptophan, tryptophan-valine, tryptophan-valine-glutamine and tryptophan-valine-glutamine-lysine
RSSTGTITTSNYANX

SEQ ID NO: 32 - protein, 8 aa, Mus musculus
  MISC_FEATURE (8)..(8): where X is selected from glycine, glycine-valine, glycine-valine-proline and glycine-valine-proline-valine
ATSIRAPX

SEQ ID NO: 33 - protein, 10 aa, Mus musculus
  MISC_FEATURE (10)..(10): where X is selected from phenylalanine, phenylalanine-glycine, phenylalanine-glycine-glycine and phenylalanine-glycine-glycine-glycine
ALWYNTHYVX

SEQ ID NO: 34 - DNA, 27 bp, Artificial Sequence
  chemically synthesized
acgtgaattcggcttaccatggcctgg

SEQ ID NO: 35 - DNA, 29 bp, Artificial Sequence
  chemically synthesized
acgtgaattctcactaacactctcccctg

SEQ ID NO: 36 - DNA, 24 bp, Artificial Sequence
  chemically synthesized
ggcttaccatggcctggacttcac

SEQ ID NO: 37 - DNA, 28 bp, Artificial Sequence
  chemically synthesized
tcactaacactctcccctgttgaagctc

SEQ ID NO: 38 - DNA, 711 bp, Artificial Sequence
  chemically synthesized
atggcctggacttcacttatactctctctcctggctctctgctcaggagccagttcccaggctgttgtgattcaggaatctgcactcaccacatcacctggtggaacagtcatactcacttgtcgctcaagtactgggactattacaactagtaactatgccaactgggtccaaaaaaaacctaatcatgtattcactggtctaataggtgctaccagcatccgagctccaggtgttcctgtcagattctcaggtttcctgattggaggcaaggctgccctcaccatcacgggggcacagactgaggatgatgcaatgtatttctgtgctctatggtacaacacccattatgttttcggcggtggaaccaaggtcactgtcctaggtcagcccaaggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 39 - protein, 237 aa, Artificial Sequence
  chemically synthesized
MAWTSLILSLLALCSGASSQAVVIQESALTTSPGGTVILTCRSSTGTITTSNYANWVQKKPNHVFTGLIGATSIRAPGVPVRFSGFLIGGKAALTITGAQTEDDAMYFCALWYNTHYVFGGGTKVTVLGQPKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 11 aa, Artificial Sequence
  chemically synthesized
YVGTGTKVTVL

SEQ ID NO: 41 - protein, 774 aa, Artificial Sequence
  chemically synthesized
GSETMEVQLVESGGGLVQPGGSLRLSCAASGFIFSSDWMNWVRQAPGKGLEWVANINQDGSEKYYVDSVKGRFTISRDNAQNSLYLQMNSLRAEDTAVYYCAKELGPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHRPRINSPTLFLSSSWALTRRPVLLDSNQEDVRMPFAEMQASFLILFYLPIYRIFFVILFLLVRACSSAYLAADEYLVVGVWENHSSLMFFLVFPTPLQSTEDVRSSFVQAYRALMRFITVKLLTQSGTVYEIQCAHRHPRHRHPGCCRHRLGYAGTAGPLAGTLSWNLIQKRDLIQDELSLVRNANGQLVKKKLPKVAIPFVLFGIDRMLKNNLINARSLSIASEPRCTCAETQMGKHPLFGLCIVSTLYASKILVGILLIARSSKFNCSNPYLTAIYKQKEAALSTFFFIIIISLLSLRLVPIDKLLILTTFNDNLRRSKNNLFEDSETMQAVVTQESALTTSPGGTVILTCRSSTGTITTSNYANWVQKKPNHVFTGLIGATSIRAPGVPVRFSGSLIGGKAALTITGAQTEDDAMYFCALWYNTHYVFGTGTKVTVLGQPKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - DNA, 2405 bp, Artificial Sequence
  chemically synthesized
ggatccgaaacgatggaagtgcagctagtcgaatccggtggaggtttggtgcaaccaggcggatcactacgactttcttgtgccgcatctggtttcatctttagttcagattggatgaattgggtcaggcaagctcccggtaaaggattggagtgggtcgctaacatcaaccaagatgggtccgaaaagtattacgttgattccgttaaaggtaggtttaccattagtagagacaacgctcagaacagtctgtatcttcagatgaactcattgagagctgaggacacagctgtttactattgtgctaaggaacttgggccttggggtcaaggaactttggtaacggtttcctctgcctctacgaaaggtccttctgtatttcccttagctccatcatcaaaatccacatctggaggtacagctgctttgggctgcctagttaaagactatttcccagaacccgtaacagtatcttggaatagtggtgccctaacaagtggagtccatacgtttccagcagtcttgcaatcctccggcttatactcattaagttctgtcgtgaccgtgccttctagttccctgggtactcaaacttacatctgtaatgtcaatcacaaaccatctaacaccaaggtagataagagagtcgaaccaaagtcatgcgataagacccattagtaacggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggataaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaagattccgaaacgatgcaggcagtggtgactcaagaatctgcattgacaacctctcccggtggaacagttatactgacgtgtcgttctagtactggaactataactacctctaactacgctaactgggttcaaaagaaaccaaatcatgttttcactggcctgattggagcaacatccattagagcccctggagttccagtgagattttctggatctttgattggtggtaaggccgctttgactattacgggtgcccaaaccgaagatgacgcaatgtacttctgcgctctttggtacaacactcattatgttttcggtaccggcactaaagttactgtgttgggtcaacctaaagtcgctgccccttcagtctttatctttcctccatccgatgagcagcttaagagtggaactgcttccgttgtttgtttgcttaacaacttttaccctcgtgaggcaaaagttcaatggaaagtcgataatgcacttcagtctggaaattctcaagagtctgttaccgagcaggattctaaggacagtacttattcactgagttcaactttgaccttgtcaaaggcagattacgaaaagcacaaagtttatgcctgcgaggtcacgcaccaggggttatcttccccagttacaaagtccttcaatcgaggggaatgttgataagcggccgc

SEQ ID NO: 43 - DNA, 2919 bp, Artificial Sequence
  chemically synthesized
atgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctgaagtgcagctagtcgaatccggtggaggtttggtgcaaccaggcggatcactacgactttcttgtgccgcatctggtttcatctttagttcagattggatgaattgggtcaggcaagctcccggtaaaggattggagtgggtcgctaacatcaaccaagatgggtccgaaaagtattacgttgattccgttaaaggtaggtttaccattagtagagacaacgctcagaacagtctgtatcttcagatgaactcattgagagctgaggacacagctgtttactattgtgctaaggaacttgggccttggggtcaaggaactttggtaacggtttcctctgcctctacgaaaggtccttctgtatttcccttagctccatcatcaaaatccacatctggaggtacagctgctttgggctgcctagttaaagactatttcccagaacccgtaacagtatcttggaatagtggtgccctaacaagtggagtccatacgtttccagcagtcttgcaatcctccggcttatactcattaagttctgtcgtgaccgtgccttctagttccctgggtactcaaacttacatctgtaatgtcaatcacaaaccatctaacaccaaggtagataagagagtcgaaccaaagtcatgcgataagacccattagtaacggccgcgaattaattcgccttagacatgactgttcctcagttcaagttgggcacttacgagaagaccggtcttgctagattctaatcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagctgatgaatatcttgtggtaggggtttgggaaaatcattcgagtttgatgtttttcttggtatttcccactcctcttcagagtacagaagattaagtgagaagttcgtttgtgcaagcttatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggataaacgctgtcttggaacctaatatgacaaaagcgtgatctcatccaagatgaactaagtttggttcgttgaaatgctaacggccagttggtcaaaaagaaacttccaaaagtcgccataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaagatccaaacgatgagatttccttcaatttttactgcagttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggctgaagctcaggcagtggtgactcaagaatctgcattgacaacctctcccggtggaacagttatactgacgtgtcgttctagtactggaactataactacctctaactacgctaactgggttcaaaagaaaccaaatcatgttttcactggcctgattggagcaacatccattagagcccctggagttccagtgagattttctggatctttgattggtggtaaggccgctttgactattacgggtgcccaaaccgaagatgacgcaatgtacttctgcgctctttggtacaacactcattatgttttcggtaccggcactaaagttactgtgttgggtcaacctaaagtcgctgccccttcagtctttatctttcctccatccgatgagcagcttaagagtggaactgcttccgttgtttgtttgcttaacaacttttaccctcgtgaggcaaaagttcaatggaaagtcgataatgcacttcagtctggaaattctcaagagtctgttaccgagcaggattctaaggacagtacttattcactgagttcaactttgaccttgtcaaaggcagattacgaaaagcacaaagtttatgcctgcgaggtcacgcaccaggggttatcttccccagttacaaagtccttcaatcgaggggaatgttgataagcggccgc

SEQ ID NO: 44 - protein, 616 aa, Artificial Sequence
  chemically synthesized
MRFPSIFTAVLFAASSALAAPVNTTTEDETAQIPAEAVIGYSDLEGDFDVAVLPFSNSTNNGLLFINTTIASIAAKEEGVSLEKREAEAEVQLVESGGGLVQPGGSLRLSCAASGFIFSSDWMNWVRQAPGKGLEWVANINQDGSEKYYVDSVKGRFTISRDNAQNSLYLQMNSLRAEDTAVYYCAKELGPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHMRFPSIFTAVLFAASSALAAPVNTTTEDETAQIPAEAVIGYSDLEGDFDVAVLPFSNSTNNGLLFINTTIASIAAKEEGVSLEKREAEAQAVVTQESALTTSPGGTVILTCRSSTGTITTSNYANWVQKKPNHVFTGLIGATSIRAPGVPVRFSGSLIGGKAALTITGAQTEDDAMYFCALWYNTHYVFGTGTKVTVLGQPKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 739 bp, Artificial Sequence
  chemically synthesized
gcggccgcggcttaccatggcctggacttcacttatactctctctcctggctctctgctcaggagccagttcccaggctgttgtgacccaggaatctgcactcaccacatcacctggtggaacagtcatactcacttgtcgctcaagtactgggactattacaactagtaactatgccaactgggtccaaaaaaaacctaatcatgtattcactggtctaataggtgctaccagcatccgagctccaggtgttcctgtcagattctcaggttccctgattggaggcaaggctgccctcaccatcacgggggcacagactgaggatgatgcaatgtatttctgtgctctatggtacaacacccattatgttttcggcacaggaaccaaggtcactgtcctaggtcagcccaaggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgagaattc

SEQ ID NO: 46 - protein, 237 aa, Artificial Sequence
  chemically synthesized
MAWTSLILSLLALCSGASSQAVVTQESALTTSPGGTVILTCRSSTGTITTSNYANWVQKKPNHVFTGLIGATSIRAPGVPVRFSGSLIGGKAALTITGAQTEDDAMYFCALWYNTHYVFGTGTKVTVLGQPKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 8 aa, Homo sapiens
  MISC_FEATURE (1)..(1): where X is selected from serine, phenylalanine-serine, isoleucine-phenylalanine-serine, and phenylalanine-isoleucine-phenylalanine-serine
XSDWMNWX

SEQ ID NO: 48 - protein, 19 aa, Homo sapiens
  MISC_FEATURE (1)..(1): where X is selected from alanine, valine-alanine, tryptophan-valine-alanine and glutamic acid-tryptophan-valine-alanine
XNINQDGSEKYYVDSVKGX

SEQ ID NO: 49 - protein, 6 aa, Homo sapiens
  MISC_FEATURE (1)..(1): where X is selected from lysine, alanine-lysine, cysteine-alanine-lysine and tyrosine-cysteine-alanine-lysine
XELGPX

SEQ ID NO: 50 - protein, 16 aa, Mus musculus
  MISC_FEATURE (1)..(1)
  MISC_FEATURE (16)..(16): where X is selected from tryptophan, tryptophan-valine, tryptophan-valine-glutamine and tryptophan-valine-glutamine-lysine
XRSSTGTITTSNYANX

SEQ ID NO: 51 - protein, 9 aa, Mus musculus
  MISC_FEATURE (1)..(1): where X is selected from glycine, isoleucine-glycine, leucine-isoleucine-glycine and glycine-leucine-isoleucine-glycine
XATSIRAPX

SEQ ID NO: 52 - protein, 11 aa, Mus musculus
  MISC_FEATURE (1)..(1): where X is selected from cysteine, phenylalanine-cysteine, tyrosine-phenylalanine-cysteine and methionine-tyrosine-phenylalanine-cysteine
XALWYNTHYVX

SEQ ID NO: 53 - DNA, 668 bp, Artificial Sequence
  chemically synthesized
caggcagtggtgactcaagaatctgcattgacaacctctcccggtggaacagttatactgacgtgtcgttctagtactggaactataactacctctaactacgctaactgggttcaaaagaaaccaaatcatgttttcactggcctgattggagcaacatccattagagcccctggagttccagtgagattttctggatctttgattggtggtaaggccgctttgactattacgggtgcccaaaccgaagatgacgcaatgtacttctgcgctctttggtacaacactcattatgttttcggtaccggcactaaagttactgtgttgggtcaacctaaagtcgctgccccttcagtctttatctttcctccatccgatgagcagcttaagagtggaactgcttccgttgtttgtttgcttaacaacttttaccctcgtgaggcaaaagttcaatggaaagtcgataatgcacttcagtctggaaattctcaagagtctgttaccgagcaggattctaaggacagtacttattcactgagttcaactttgaccttgtcaaaggcagattacgaaaagcacaaagtttatgcctgcgaggtcacgcaccaggggttatcttccccagttacaaagtccttcaatcgaggggaatgttgataagcggccgc